(19) United States
(12) Patent Application Publication
(10) Pub. No.: US 2008/0207532 A1
(43) Pub. Date: Aug. 28, 2008

(54) Use of Nordihydroguaiaretic Acid Derivatives in the Treatment of Drug Resistant Cancer, Viral and Microbial Infection

(75) Inventors: Ru Chih Huang, Baltimore, MD (US); Jih Ru Hwu, Hsinchu (TW); Ming-Hua Hsu, Hsinchu (TW); David E. Mold, Baltimore, MD (US); Yuan C. Lee, Baltimore, MD (US); Chih-Chuan Chang, Baltimore, MD (US)

Correspondence Address: VENABLE LLP, P.O. BOX 34385, WASHINGTON, DC, 20043-9998, US

(73) Assignee: Johns Hopkins University, Baltimore, MD (US)

(21) Appl. No.: 11/664,875

(22) PCT Filed: Oct. 6, 2005
(86) PCT No.: PCT/US2005/035795

§ 371 Date: Apr. 15, 2008

Related U.S. Application Data
(60) Provisional application No. 60/616,114, filed on Oct. 6, 2004.

(51) Int. Cl. A61K 31/7034; C12N 5/06; C07H 15/04; C12Q 1/02; A61P 35/00; A61P 31/00

(52) U.S. Cl. 514/25; 435/375; 536/4.1; 435/29

(57) ABSTRACT

Compositions and methods for using nordihydroguaiaretic acid (NDGA) derivatives for preventing the expression of MDR-1 gene and the synthesis of PgP protein or reversing multiple drug resistance in cells, and for using NDGA derivatives in combination with additional chemotherapeutic agents to treat drug resistant cancer and infections.

### BACKGROUND OF THE INVENTION

1. Field of the Invention

The invention relates to compounds and methods for preventing or reversing multiple drug resistance in cells.

2. Background Information

A synthetic derivative of the naturally occurring plant lignan, tetra-o-methyl nordihydroguaiaretic acid (tetra-o-methyl NDGA or M₄N), was found to possess antiviral and anticancer activities, not by binding to essential viral or cell cycle related proteins, but by blocking the transcription of these growth related genes in a mutation insensitive way, affording M₄N effectiveness for the long term use (1-3, 15). M₄N, both in cell cultures and in five human cancer xenografts in Thy⁻/Thy⁻ mice (breast cancer, MCF-7, liver cancer Hep3B, colorectal carcinoma HT-29, prostate carcinoma LNCaP and chronic myelogenous leukemia K562) is able to inhibit SP₁-regulated Cdc2 and survivin gene expressions which consequently induces cell arrest at the G₂/M phase of the cell cycle and apoptosis in a timely manner (4, 5). During the initial stage of this process, M₄N treated cancer cells can regain their replicative and antiapoptotic capabilities if these cells were transfected with a SP₁-independent, CMV promoter-driven construct of this pair of genes in the presence of M₄N. The discovery conforms to the model that the effect of M₄N on transcription of these two genes is promoter SP₁-dependent (4). Upon injection by IP and IV routes and through oral feeding of M₄N, the distribution and accumulation of M₄N in mouse tissues were found to be selective and the level of drug in the blood was extremely low and only presents transiently (5). The amount was also barely detectable in dogs and rabbits in all toxicity studies using subcutaneous, intravaginal and IV formulations. In all toxicology studies so far conducted, no evidence was observed of the type of progenitor cell eliminations seen, for example, with cisplatin or other non-selective cytotoxics. In clinical trials to date, M₄N has not caused the systemic side effects such as gastrointestinal problems, anemia and hair loss (6).

Multiple drug resistant gene 1 (MDR1) encodes a 170 kda membrane protein ATPase P-glycoprotein (Pgp) drug transporter (7). MDR1 is one member of the ATP-binding cassette (ABC) family (8) commonly known for its ability to expel cytotoxic compounds following chemotherapeutic treatments (9). Substrates for Pgp protein include drugs such as doxorubicin (Dox) (also known as adriamycin), vinblastine, paclitaxel, vincristine, and many others (10). Mechanisms underlying the MDR-1 gene expression have been extensively studied recently. In response to environmental signals, high levels of MDR-1 expression were found to depend upon a group of transcription factors interacting with unique DNA sequences at the MDR1 promoter site in a form of an enhancesome. Upon Dox induction, transcription of MDR-1 proceeds quickly. During this process, it has been suggested that transcription factor NF-Y1 joins Sp₁/Sp₃ to recruit P/CAF histomeacetyltransferase to the MDR promoter site. Histone acetylation farther facilitates the formation of a local chromatin structure that is compatible for active MDR1 transcription (10). The search for compounds that can inhibit MDR1 and Pgp synthesis has been progressing actively in many research laboratories. For example, it has been found that compound ET-743, which targets NF-Y1/PCAF complex, is able to inhibit MDR-1 gene activation (11). Repressor K2-5F which competes with Sp₁/EGR₁/WT₁ for binding the GC elements of the MDR-1 promoter significantly reduced MDR-1 expression (12).

Drug resistance is one of the major problems associated with cancer treatments that use cytotoxic drugs such as Dox, vinblastine, paclitaxel, vincristine and others. Long term use of Dox, for example, causes drug resistance to occur in primary and in mestastatic tumors resulting from high MDR-1 gene expression and accumulation of the cellular ATPase drug transporter protein Pgp. Pgp acts to expel drugs from the tumor cells resulting in less of the drug being situated where it is able to inhibit tumor growth.

### SUMMARY

The invention includes, inter alia, compositions, uses and methods for treating drug resistance (e.g. multiple drug resistance) in cells and organisms, and for treating cancer and other diseases wherein cells or microorganisms have developed, or may develop, resistance to one or more chemotherapeutic agent or drug.

Accordingly, the invention includes use of an NDGA derivative or physiologically acceptable salt thereof for preventing at least one of synthesis and function of drug transporter protein Pgp in a cell; the NDGA derivative having the formula

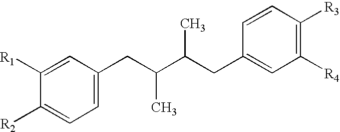

wherein R₁, R₂, R₃ and R₄ are each independently selected from the group consisting of HO—, CH₃ O—, CH₃(C═O)O—, an amino acid residue, a substituted amino acid residue and a saccharide residue; the amino acid residue, substituted amino acid residue or saccharide residue being optionally joined to the phenyl ring by a linker of an oxygen atom and 1-10 carbon atoms, with the proviso that

i) at least one of R₁-R₄ comprises an amino acid residue or substituted amino acid residue, or

ii) one of R₁-R₄ comprises a saccharide residue.

The invention also includes use of an NDGA derivative or physiologically acceptable salt thereof in combination with at least one secondary chemotherapeutic agent to treat cancer, the NDGA derivative having a formula

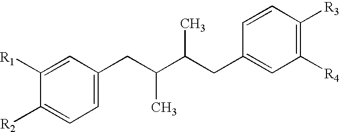

wherein

i) at least one of R₁-R₄ comprises an amino acid residue having at least 2 —CH₂— groups present between an amino and a carboxyl group, or comprises a substituted amino acid residue having at least 2 —CH₂— groups between an amino and a carboxyl group, and the remaining R groups are independently selected from HO—, CH₃ O— and CH₃(C═O)O—; or

ii) one of R₁-R₄ comprises a saccharide residue and the remaining R groups are selected from HO—, CH₃ O— and CH₃(C═O)O—;

the amino acid residue, substituted amino acid residue or saccharide residue being optionally joined to the phenyl ring by a linker of an oxygen atom and 1-10 carbon atoms.

The invention further includes use of an NDGA derivative or physiologically acceptable salt thereof to prevent or overcome multiple drug resistance in a cancer cell; the NDGA derivative having a formula

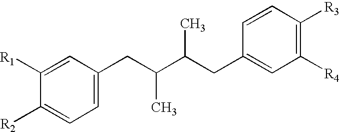

wherein R₁, R₂, R₃ and R₄ are each independently selected from the group consisting of HO—, CH₃ O—, CH₃(C═O)O—, an amino acid residue, a substituted amino acid residue and a saccharide residue; the amino acid residue, substituted amino acid residue or saccharide residue being optionally joined to the phenyl ring by a linker of an oxygen atom and 1-10 carbon atoms, with the proviso that

i) at least one of R₁-R₄ comprises an amino acid residue or substituted amino acid residue, or

ii) one of R₁-R₄ comprises a saccharide residue.

Also included is a method of overcoming drug resistance in a microorganism, comprising administering to said microorganism or a host containing the microorganism an effective amount of an NDGA derivative or physiologically acceptable salt thereof, wherein the NDGA derivative has a formula

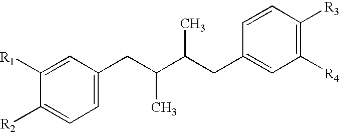

wherein R₁, R₂, R₃ and R₄ are each independently selected from the group consisting of HO—, CH₃ O—, CH₃(C═O)O—, an amino acid residue, a substituted amino acid residue and a saccharide residue; the amino acid residue, substituted amino acid residue or saccharide residue being optionally joined to the phenyl ring by a linker of 1-10 carbon atoms; with the proviso that

i) at least one of R₁-R₄ comprises an amino acid residue or substituted amino acid residue, or

ii) one of R₁-R₄ comprises a saccharide residue.

Furthermore, the invention includes a method of treating a drug-resistant infection in an animal, comprising administering to the animal, along with at least one therapeutic agent to which the infection is resistant, an effective amount of a compound or a physiologically acceptable salt of the compound, the compound having a formula

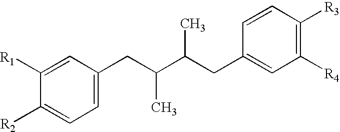

wherein R₁, R₂, R₃ and R₄ are each independently selected from the group consisting of HO—, CH₃ O—, CH₃(C═O)O—, an amino acid residue, a substituted amino acid-residue and a saccharide residue; the amino acid residue, substituted amino acid residue or saccharide residue being optionally joined to the phenyl ring by a linker of 1-10 carbon atoms, with the proviso that

i) at least one of R₁-R₄ comprises an amino acid residue or substituted amino acid residue, or

ii) one of R₁-R₄ comprises a saccharide residue.

The invention includes a composition comprising a compound or a physiologically acceptable salt of the compound, the compound having a formula

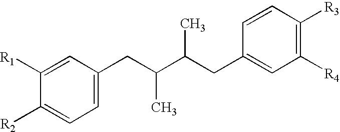

wherein

i) at least one of R₁-R₄ is a β-amino acid residue linked to the phenyl ring through an oxygen atom; or

ii) one of R₁-R₄ a saccharide residue, optionally linked to the phenyl ring through an oxygen atom and 1-10 —CH₂-groups; and the remaining R groups are —OCH₃.

Furthermore, the invention includes a composition comprising an NDGA derivative, or a physiologically acceptable salt thereof, and a secondary chemotherapeutic agent, the NDGA derivative having a formula

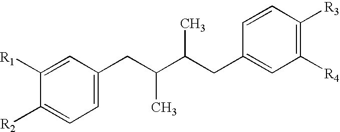

wherein

i) at least one of R₁-R₄ comprises an amino acid residue having at least 2 —CH₂— groups present between an amino and a carboxyl group, or comprises a substituted amino acid residue having at least 2 —CH₂— groups between an amino and a carboxyl group, and the remaining R groups are independently selected from HO—, CH₃ O— and CH₃(C═O)O—; or

ii) one of R₁-R₄ comprises a saccharide residue and the remaining R groups are selected from HO—, CH₃ O— and CH₃(C═O)O—;

the amino acid residue, substituted amino acid residue or saccharide residue being optionally joined to the phenyl ring by a linker of an oxygen atom and 1-10 carbon atoms.

The invention also includes a method for determining an optimum dosage combination of an NDGA derivative or physiologically acceptable salt thereof, wherein the NDGA derivative has a formula

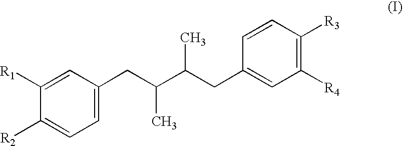

wherein R₁, R₂, R₃ and R₄ are each independently selected from the group consisting of HO—; CH₃ O—; CH₃(C═O)O—; an amino acid residue; a substituted amino acid residue; a saccharide residue; the amino acid residue, substituted amino acid residue or saccharide residue being optionally joined to the phenyl ring by a linker of 1-10 carbon atoms and an oxygen atom; and a secondary chemotherapeutic agent for treating a cancer, comprising

(a) administering a series of compositions of varying dosages of the NDGA derivative or physiologically acceptable salt thereof and the secondary chemotherapeutic agent to a culture of cancer cells;

(b) measuring the growth rate of the cells;

(c) using an isobologram method or combination index method to determine the optimal combination dosage to achieve comparable efficacy with suboptimal concentrations for both the NDGA derivative or physiologically acceptable salt thereof and the secondary chemotherapeutic agent.

These and other aspects of the invention are described more fully described in the following sections and in the claims.

### BRIEF DESCRIPTION OF THE DRAWINGS

FIG. 1. Effect of M₄N treatment on growth of MCF cells and NCI/ADR-RES cells. A. In culture. B. In xenografts of Thy⁻/Thy⁻ mice.

FIG. 2. Effect of M₄N treatment on Cdc2 and survivin production in MCF-7 and NCI/ADR cells—Western blot analysis.

FIG. 3. Dose-effect curves for M₄N, Dox, paclitaxel and their combinations in NCI/ADR-RES cells. A. M₄N and doxorubicin (Dx), alone and in combination. B. M₄N and paclitaxel (Px), alone and in combination. The x-axis represents the dose of drug in μmoles/liter and the y-axis represents Fa, the fraction of cells affected (growth inhibition).

FIG. 4. Analysis of the combination of M₄N with Dox or paclitaxel in NCI/ADR-RES cells. A. Isobologram for the combination of M₄N with doxorubicin (Dx) at different effect levels (Fa). B. Isobologram for the combination of M₄N with paclitaxel (Px) at different effect levels (Fa).

FIG. 5. Analysis of the combination of M₄N with Dox or paclitaxel in NCI/ADR-RES cells. A. CI plot for the combination of M₄N with doxorubicin (Dx). B. CI plot for the combination of M₄N and paclitaxel (Px).

FIG. 6. Effect of M₄N on MDR1 gene expression and Pgp levels in NCI/ADR-RES human breast cancer cells. A. Agarose gel analysis of MDR1 and GAPDH (normalization control) cDNAs generated by RT-PCR of total RNA from cells treated for three days with 0, 5, 10 and 20 μM M₄N. Results in bar graph form normalized to GAPDH. B. Western blot analysis of Pgp and cyclin B1 (normalization control) protein levels in cells treated for three days with 0, 5, 10 and 20 μM M₄N. Results in bar graph form normalized to cyclin B1.

FIG. 7. Effect of M₄N on induction of MDR1 gene expression by doxorubicin in MCF-7 cells. MCF-7 cells were left untreated or treated with 0.05 μM doxorubicin in the presence or absence of 5 μM M₄N for two days and then total RNA and protein were analyzed for MDR1 gene expression and Pgp protein levels. A. Agarose gel analysis of MDR1 and GAPDH (normalization control) cDNAs generated by RT-PCR. STD, cDNAs from NCI/ADR-RES cells. B. Western blot analysis of Pgp and cyclin B1 (normalization control) protein levels. STD, analysis of proteins from NCI/ADR-RES cells.

FIG. 8. Inhibition of Pgp-mediated Efflux of Rhodamine 123. NCI/ADR-RES cells, incubated for three days in the presence of 0, 1.25, 2.5, 3.75 and 5.0 μM M₄N, were tested for their ability to retain Rhodamine 123. The percent Rhodamine 123 remaining in the cells was plotted against time.

FIG. 9. (A) Effect of Maltose-M₃N on the proliferation of human tumor cell lines. HT29, LNCaP, Hep3B, K562, and MCF7 were treated with different concentrations of Maltose-M₃N for 72 hours. Percent cell viability was measured by MTT assay and expressed as mean±SD of triplicate data points. (B) Untreated Hep3B cells or treated with 60 μM Maltose-M₃N for 72 hr were stained with DAPI. Arrows indicate apoptotic bodies.

FIG. 10. Effect of Maltose-M₃N on Cdc2 and survivin protein expression in human tumor cell lines. Total protein extracts were prepared from control cells [C] or cells exposed to Maltose-M₃N [M] for 24 and 72 hours and analyzed for CDC2 and survivin levels by Western blot analysis. β-actin was used as a loading control.

FIG. 11. Effect of intratumoral Maltose-M₃N treatment on apoptosis, and CDC2 and survivin protein levels of C3 tumors. Tumors were excised from mice treated daily for 4 days with intratumoral injections of the indicated concentrations of Maltose-M₃N or placebo (0.15 M NaCl). Fixed tumors were sectioned and analyzed by H&E staining and immunochemical analysis using antibodies specific for CDC2 and survivin.

FIG. 12. Effect of M₄N and maltose M₃N (Mal-M₃N) alone and in combination with Paclitaxel (Px), on Pgp protein levels of NCI/ADR-RES breast cancer xenograft tumors in nude mice. Formaldehyde fixed tumors from mice treated daily for two weeks with i.p. injections of M₄N (320 μmol/m²), Mal-M₃N (320 μmol/m²) or Px (16 μmol/m²), alone or in combination, were sectioned and analyzed by H&E and immunochemical staining using antibodies specific for human Pgp.

### DESCRIPTION OF THE INVENTION

The present invention makes use of nordihydroguaiaretic acid derivatives, such as tetra-o-methyl NDGA, (M₄N) to stop the formation and/or the function of Pgp protein. The inventors have found that M₄N inhibits Dox-induced MDR gene expression, synthesis of Pgp protein and prevents the “pump-out” of Dox from drug treated cells. M₄N treatment makes Dox more available for targeting TopII/DNA complex at the S phase of the cell cycle. Thus, low concentrations of M₄N and Dox can be used synergistically to control the cancer growth.

Furthermore, it was found that M₄N and derivatives thereof are extremely effective in elimination of human breast cancer xenografts of NCI/ADR-RES cells, a cell line that has already acquired strong resistance to Dox. M₄N blocks Cdc2 and survivin gene expressions in NCI/ADR-RES cells and remarkably M₄N is able to prevent the expression of MDR1 gene as well. Other NDGA derivatives, as described below, should also exhibit these properties.

By “nordihydroguaiaretic acid derivatives”, as used herein, is meant compounds of the structure

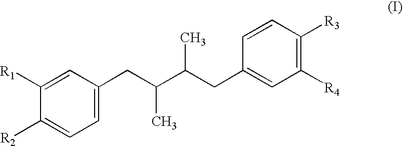

wherein R₁, R₂, R₃ and R₄ are each independently selected from the group consisting of HO—, CH₃ O— and CH₃(C═O)O—, provided that R₁, R₂, R₃ and R₄ are not each HO— simultaneously; an amino acid residue; a substituted amino acid residue; and physiologically acceptable salts thereof. Also included are compounds of this formula wherein one of R₁, R₂, R₃ and R₄ is a saccharide residue and the remaining R groups are independently selected from HO—, CH₃ O— and CH₃(C═O)O—, and physiologically acceptable salts thereof. The amino acid residue, substituted amino acid residue or saccharide residue can optionally be joined to the phenyl ring by a linker of 1-10, more usually 1-6, carbon atoms, most usually with an oxygen atom linking the carbon linker to the phenyl ring. Typically the linker includes 2-4 —CH₂— groups, e.g. (saccharide residue)—CH₂—CH₂—O—(phenyl). R₁-R₄ may be identical or different, except in certain applications (e.g. treatment of cancer), wherein R₁, R₂, R₃ and R₄ may not each be HO— simultaneously. It is preferred that at least one of R₁-R₄ comprise an amino acid residue; a substituted amino acid residue; a saccharide residue.

Amino acids to be used as substituents include both naturally occurring and synthetic amino acids. These include, inter alia alanine, arginine, asparagine, aspartate, cysteine, glutamate, gluamine, glycine, histidine, isoleucine, leucine, lysine, methionine, phenylalanine, proline, serine, threonine, tryptophan, tyrosine, valine, 5-hydroxylysine, 4-hydroxyproline, thyroxine, 3-methylhistidine, ε-N-methyllysine, ε-N,N,N-trimethyllysine, aminoadipic acid, γ-carboxyglutamic acid, phosphoserine, phosphothreonine, phosphotyrosine, N-methylarginine, and N-acetyllysine. Both R and L forms and mixed R and L forms of alpha amino acids are contemplated. Further possible substituents include amino acids that have 2 or more —CH₂— groups (usually 2-4) present between the amino and carboxyl groups, as described in more detail below, and derivatives thereof. Thus, the amino acid residue may be the residue of an alpha, beta, gamma, or higher order amino acid, preferably one corresponding to a naturally occurring amino acid in other respects. Amino acid residues are preferably linked to the phenyl ring through an oxygen atom joined to a carbonyl group in the residue.

Substituted amino acid residues and derivatives of amino acids are intended to include those residues wherein one or more hydrogen atoms have been replaced by a methyl or other straight or branched chain lower alkyl group (1-6 carbon atoms), a sulphate or phosphate group, etc., for example, wherein a dimethyl substitution is made on the amino group.

Because of the limited solubility of M₄N in aqueous solution, there is a further need for water soluble compounds having similar properties in order to more easily formulate and administer pharmaceutical compositions. Improvement of solubility can be achieved, for example, by glycosylation of M₃N to obtain a compound wherein one of R₁-R₄ is a saccharide, e.g. a monosaccharide or a disaccharide. For steric reasons, it has been found preferable to include a linking group comprising 1-10, more usually 1-6, carbon atoms between the saccharide and an oxygen atom linked to the phenyl group, generally 2-4 —CH₂— groups. Examples of saccharides that are useful are maltose, galactose, mannose, fucose, glucosamine and their derivatives in which the OH groups of the sugars are replaced by other groups such as O-methyl, O-acetyl, amino, carboxyl, lower alkyl, lower acyl (of 1 to 6 carbon atoms), phospho and/or sulfo groups, or oligosaccharides of 2 or more sugars of the same or different kinds. Examples of such compounds are those wherein R₁ or R₂ is maltose or galactose and the remaining R groups are —OCH₃, such as the following compounds (designated galactose-M₃N and maltose-M₃N, respectively):

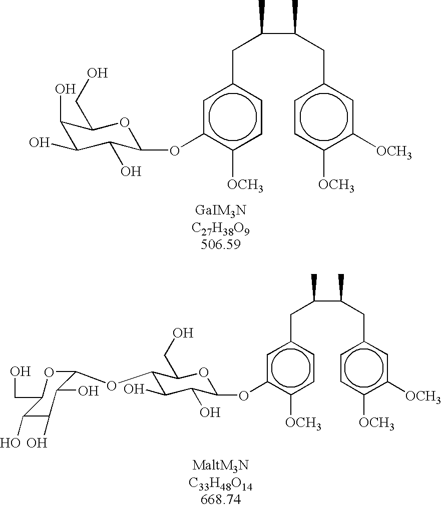

Tumors to be treated include any cancerous or noncancerous tumor that exhibits drug resistance, in particular drug resistance caused by the presence/overexpression of an MDR1 gene in the cells (Ling, V. Multidrug resistance: Molecular Mechanisms and Clinical Relevance, Cancer Chemother. Pharmacol. 40:53-58, 1997). Such tumors include, inter alia, breast cancer, metastatic carcinoma of the lung, primary melanoma, ovarian cancer, multiple myeloma, and Non-Hodgkin's Lymphoma.

The term “cancerous tumor” is intended to include any malignant tumor that may or may not have undergone metastasis. The term “noncancerous tumor” is intended to include any benign tumor. These terms are used as customarily understood by persons of skill in the art.

Additional examples of benign and malignant tumors which may be treated by the compositions and methods of the invention can be found in Table 1-1 of Cancer Biology (Raymond W. Ruddon, Cancer Biology, 3rd Ed., Oxford Univ. Press, 1995, incorporated herein by reference). Tumors to be treated include those that are known to be of viral origin, as well as those that are not of viral origin. The compositions and methods of the invention are expected to be particularly useful in the treatment of solid tumors.

By “multidrug resistant” or “MDR” is meant cells, microorganisms, etc. that are resistant to one or more therapeutic compounds intended to inactivate or kill those cells or microorganisms, including those that, for example, exhibit high MDR-1 gene expression. The term “drug resistant” is also used to refer to cells, microorganisms, etc., that are known to be resistant to a particular therapeutic compound.

It is contemplated that the NDGA derivatives and pharmaceutical compositions comprising the derivatives will be administered locally (e.g. topically or by local injection into the tumors), or by systemic delivery (e.g. orally, intraperitoneally, intravenously, subcutaneously or intramuscularly) generally along with pharmaceutically acceptable diluents, excipients and carriers. Non water-soluble derivatives may be formulated into pharmaceutical compositions in suitable solvents for injection into tumors, for example in the form of a DMSO solution. Other means of local administration, such as topical application or targeted delivery to the tumor site, may also be used.

Compounds and compositions that are water soluble may be formulated in any pharmaceutically acceptable aqueous solution, for example a phosphate buffer solution (PBS). NDGA derivatives may also be employed in lipid based or water based formulations for systemic delivery, as known and used in the art.

The NDGA derivatives may be used as non-polar compounds or in the form of a free acid/base, or in the form of a tetrahydrochloride, or other physiologically acceptable salt.

The compounds of the invention may be used in combination with other diagnostic or therapeutic compounds and with pharmaceutically acceptable diluents, excipients and carriers, as will be clear to those of skill in the art.

By “pharmaceutically acceptable diluents, excipients and carriers” is meant such compounds as will be known to persons of skill in the art as being compatible with the NDGA derivatives and suitable for local or systemic administration to an animal, particularly a human or other mammal, according to the invention. Useful solutions for oral or parenteral administration can be prepared by any of the methods well known in the pharmaceutical arts, described, for example, in Remington's Pharmaceutical Sciences, (Gennaro, A., ed.), Mack Pub., (1990).

The amount of compound administered to obtain the desired treatment effect will vary but can be readily determined by persons of skill in the art. The amount of dosage, frequency of administration, and length of treatment are dependent on the circumstances, primarily on the size and type of tumor. Typical dosages are expected to be in the range of about 10-10⁴ μmoles/m², more usually 102-103 μmoles/m² of a patient's or a subject's body surface area.

In addition to being effective anticancer agents in their own right (U.S. Pat. Nos. 6,214,874, 6,417,234, 6,608,108), NDGA derivatives appear able to solve the drug resistance problem associated with many cytotoxic drugs commonly used in clinics. High efficacy of cancer control can be achieved by using these relatively nontoxic derivatives and other cytotoxic drugs jointly in low concentrations. The NDGA derivatives may thus be used as primary chemotherapeutic agents with a variety of cytotoxic agents that are used as chemotherapeutic agents for cancerous or benign tumors, for example, Dox, vinblastine, paclitaxel, and vincristine. As used herein, such additional chemotherapeutic agents will be referred to as “secondary” chemotherapeutic agents. When in combination with NDGA derivatives, reduced concentrations of these secondary chemotherapeutic agents are sufficient to achieve high efficacy. A listing of additional commonly used chemotherapeutic agents to be included in the meaning of “secondary chemotherapeutic agents” as used herein can be found in Blagosklonny, Cell Cycle 3: e52-e59 (2004); other cytotoxic compounds will be known to those of skill in the art.

The NDGA derivatives described herein may also be used for treatment of resistant viral, bacterial and other similar infections, by administration of these derivatives in combination with pharmaceutical compounds to which the microorganisms have become resistant due to the presence or overexpression of drug resistance genes.

Thus, the invention provides the use of the use of an NDGA derivative for preventing at least one of synthesis and function of drug transporter protein Pgp in a cell, the NDGA derivative having a formula

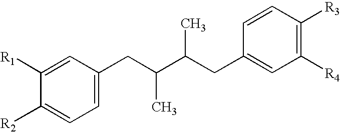

wherein R₁, R₂, R₃ and R₄ are each independently selected from the group consisting of HO—, CH₃ O— and CH₃(C═O)O—, provided that R₁, R₂, R₃ and R₄ are not each HO— simultaneously; an amino acid residue; a substituted amino acid residue; and physiologically acceptable salts of the derivative. Also included for this use are compounds of this formula wherein one of R₁, R₂, R₃ and R₄ is a saccharide residue and the remaining R groups are independently selected from HO—, CH₃ O— and CH₃(C═O)O—, and physiologically acceptable salts of the compound. The amino acid residue, substituted amino acid residue or saccharide residue are optionally joined to the phenyl ring by a linker of 1-10, more usually 1-6, carbon atoms, and an oxygen atom, as noted above. It is preferred that at least one of R₁-R₄ comprise an amino acid residue; a substituted amino acid residue; and a saccharide residue.

The amino acid residue may be, for example, a residue of a naturally occurring amino acid, or a derivative thereof, or a residue of an amino acid having at least two —CH₂— groups present between the amino group and the carboxyl group, or a derivative thereof. In one embodiment, tetra-o-methyl nordihydroguaiaretic acid (M₄N) is used. Chemical induction may be caused, for example, by chemotherapeutic agents such as Dox, vinblastine, paclitaxel, and vincristine. The cell may be, for example, a tumor cell, or an infectious microorganism, such as a virus, bacterium, parasite, or fungus.

The synthesis or function of Pgp may be prevented, e.g., by inhibition of chemical induction, preventing transcription of MDR1 and/or preventing synthesis of Pgp in a cell. The cell may be, for example, a tumor cell, or an infectious microorganism, such as a bacterium, virus, parasite or fungus.

Correspondingly, the invention provides a method for preventing at least one of synthesis and function of drug transporter protein Pgp in a cell, the method comprising administering an effective amount of one of the above-mentioned NDGA derivatives to a cell.

The above-mentioned derivatives may also be used in combination with one or more other chemotherapeutic agents, to treat cancer. These secondary chemotherapeutic agents may be, for example, Dox, vinblastine, paclitaxel, or vincristine. It has been found that specific ratios of about 2:1 to about 100:1, more often about 10:1 to about 50:1 (e.g. about 20:1), of NDGA derivative to secondary chemotherapeutic agent are particularly advantageous, as they provide optimal results for minimum dosages of each agent. In this context, “about” means±25%, i.e. ratios of 15:1 to 24:1 for a ratio of about 20:1, for instance.

Accordingly, it will be clear that the NDGA derivatives described above are useful to prevent or overcome multiple drug resistance in cancer cells, and that the invention provides a method and use for overcoming drug resistance in cancer cells, comprising administering an effective amount of the above-mentioned NDGA derivatives to the cancer cells for preventing or overcoming MDR. The drug resistance may be, for example, resistance to Dox, vinblastine, paclitaxel, vincristine, as well as other drugs that are used to treat cancer.

Thus, the invention also provides a method of treating cancer, comprising administering an NDGA derivative or physiologically acceptable salt thereof, wherein the NDGA derivative has a formula

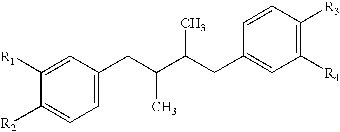

wherein

i) at least one of R₁-R₄ comprises an amino acid residue having at least 2 —CH₂— groups present between an amino and a carboxyl group, or comprises a substituted amino acid residue having at least 2 —CH₂— groups between an amino and a carboxyl group, and the remaining R groups are independently selected from HO—, CH₃ O— and CH₃(C═O)O—; or

ii) one of R₁-R₄ comprises a saccharide residue and the remaining R groups are selected from HO—, CH₃ O— and CH₃(C═O)O—; the amino acid residue, substituted amino acid residue or saccharide residue being optionally joined to the phenyl ring by a linker of 1-10 carbon atoms and an oxygen atom;

in combination with a secondary chemotherapeutic agent, to treat cancer.

Such secondary chemotherapeutic agents may be, for example, Dox, vinblastine, paclitaxel, and/or vincristine. The cancer may be a human cancer, for example, breast cancer, lung cancer, melanoma, ovarian cancer, multiple myeloma, and Non-Hodgkin's Lymphoma or may be cancer in a nonhuman animal, especially a mammal.

The invention also provides a method of overcoming drug resistance in a microorganism, comprising administering to said microorganism or a host containing the microorganism an effective amount of an NDGA derivative or physiologically acceptable salt thereof, wherein the NDGA derivative has a formula

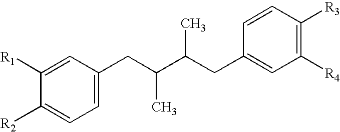

wherein R₁, R₂, R₃ and R₄ are each independently selected from the group consisting of HO—, CH₃ O—, CH₃(C═O)O—, an amino acid residue, a substituted amino acid residue and a saccharide residue; the amino acid residue, substituted amino acid residue or saccharide residue being optionally joined to the phenyl ring by a linker of 1-10 carbon atoms; with the proviso that

i) at least one of R₁-R₄ comprises an amino acid residue or substituted amino acid residue, or

ii) one of R₁-R₄ comprises a saccharide residue.

The microorganism may be, for example, a virus, a bacterium, a parasite or a fungus.

Thus, the invention also provides a method of treating a drug-resistant infection in an animal, comprising administering an effective amount of an NDGA derivative as described above to the animal, along with at least one drug (e.g. antibiotic) to which the infection is resistant. The infection may be, for example, a viral, bacterial, parasitic or fungal infection.

The invention also provides compounds and compositions to be used for the above-mentioned purposes. In particular, the invention provides a compound or a physiologically acceptable salt of the compound, the compound having a formula

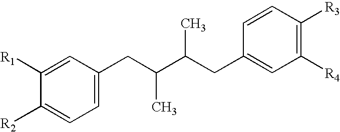

wherein

i) at least one of R₁-R₄ comprises a β-, γ- or higher order amino acid residue linked to the phenyl ring through an oxygen atom; or

ii) one of R₁-R₄ a saccharide residue, optionally linked to the phenyl ring through an oxygen atom and 1-10 —CH₂— groups; and

the remaining R groups are —OCH₃.

In one embodiment of this aspect of the invention, at least one of R₁-R₄ is a β-, γ- or higher order amino acid residue, optionally linked to the phenyl ring through an oxygen atom and 1-10 —CH₂— groups. The remaining R groups may be, for example, HO—, CH₃ O— or CH₃(C═O)O—. In another embodiment, one of R₁-R₄ is a saccharide moiety, and the remaining groups are independently selected from HO—, CH₃ O— or CH₃(C═O)O—.

In one embodiment, at least one of R₁-R₄ is a β-amino acid, and the remaining R groups are CH₃O—. β- and γ-amino acids refer to amino acids that have 2 or 3 —CH₂— groups, respectively, between the amino and carboxyl groups. In one embodiment, β- and γ-amino acid residues correspond to naturally occurring amino acids with one or two additional —CH₂— groups, respectively, present between the amino and carboxyl groups.

In one embodiment, one of R₁-R₄ is a mono-or disaccharide, for example, galactose or maltose. Examples of derivatives of these types are maltose-M₃N and galactose-M₃N, as described above, maltose —CH₂—CH₂—O—M₃N, and galactose —CH₂—CH₂—O—M₃N.

Additional examples of compounds of the invention are 5-((2S,3R)-4-{3,4-bis[4-(dimethylamino)butanoyloxy]phenyl}-2,3-dimethylbutyl)-2-[4-(dimethylamino)butanoyloxy]phenyl 4-(dimethylamino)butanoate and 5-((2S,3R)-4-{3,4-bis[4-(dimethylamino)propanoyloxy]phenyl}-2,3-dimethylbutyl)-2-[4-(dimethylamino)propanoyloxy]phenyl 4-(dimethylamino)propanoate.

These and other compounds of the invention may be formulated into compositions optionally with secondary chemotherapeutic agents, antibiotics and/or pharmaceutically acceptable excipients or carriers. In this regard, it has been found that certain combinations of NDGA derivatives and other chemotherapeutic agents can be optimally delivered in a specific molar ratio, e.g. from about 2:1 to about 100:1, for example about 20:1, with predetermined synergy of suboptimal concentrations for both compounds. Accordingly, in one embodiment, the pharmaceutical compositions of the invention are formulated in these preferred ratios, e.g. 2.4:1, 20:1, 50:1.

Accordingly, also included in the invention is a composition comprising an NDGA derivative, or a physiologically acceptable salt thereof, and a secondary chemotherapeutic agent, the NDGA derivative having a formula

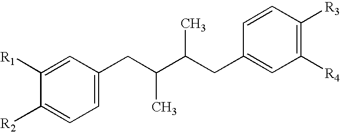

wherein

i) at least one of R₁-R₄ comprises an amino acid residue having at least 2 —CH₂— groups present between an amino and a carboxyl group, or comprises a substituted amino acid residue having at least 2 —CH₂— groups between an amino and a carboxyl group, and the remaining R groups are independently selected from HO—, CH₃ O— and CH₃(C═O)O—; or

ii) one of R₁-R₄ comprises a saccharide residue and the remaining R groups are selected from HO—, CH₃ O— and CH₃(C═O)O—;

the amino acid residue, substituted amino acid residue or saccharide residue being optionally joined to the phenyl ring by a linker of an oxygen atom and 1-10 carbon atoms.

Another aspect of the invention provides a method for determining such advantageous ratios of NDGA derivatives and other chemotherapeutic agents, and compositions comprising NDGA derivatives or physiologically acceptable salt thereof and secondary chemotherapeutic agents in these ratios. The method comprises the steps of

(a) administering a series of compositions of varying dosages of an NDGA derivative or physiologically acceptable salt thereof and a secondary chemotherapeutic agent to a culture of cancer cells;

(b) measuring the growth rate of said cells;

(c) using an isobologram method or combination index method to determine optimal combination dosages to achieve comparable efficacy with suboptimal concentrations for both the NDGA derivative or physiologically acceptable salt thereof and the secondary chemotherapeutic agent; and

(d) formulating a composition comprising the optimal dosages.

Preferably the optimally formulated composition is administered to a patient in need of treatment.

For example, an optimal combination of maltose-M₃N or M₄N with paclitaxel has been found to be 320 μmoles/m²:16 μmoles/m².

This application claims priority to U.S. provisional application No. 60/616,114, which is incorporated herein by reference.

### EXAMPLES

The present invention will now be described in more detail with reference to the following specific, non-limiting examples.

The examples below demonstrate that M₄N and other NDGA derivatives can control the synthesis of Pgp by blocking Sp₁-regulated MDR-1 gene expression. The results show that M₄N is effective in preventing Dox induction of MDR-1 gene and maintaining drug sensitivity of MCF-7 cells. M₄N and Dox showed synergistic effect in suppression of MCF-7 cell growth. Furthermore, cell growth, MDR gene expression and synthesis of Pgp protein in NCI/ADR-RES cells were all greatly reduced following M₄N treatment. In addition, the examples show that combinations of NDGA derivatives and secondary chemotherapeutic agents are synergistic and are highly effective in reducing tumor growth in vivo.

### Example 1

### M₄N Treatment Blocks Cellular Proliferation of MCF-7 and NCI/ADR Cells in Culture and in Xenografts of Thy⁻/Thy⁻ Mice

### Methods

MCF-7 cells (a human mammary carcinoma cell line, obtainable from the ATCC P.O. Box 1549, Manassas, Va. 20108) were seeded into 24-well plates at 1.5×10⁴ cells/well in 500 μl of Dulbecco's modified Eagle's medium (DMEM) containing 10% fetal bovine serum (FBS) and the antibiotics penicillin and streptomycin. Varying concentrations of M₄N were added the next day. After incubation for an additional 3 days, cell proliferation was assessed by the MTT assay. For the xenograft study, female athymic nude (nu/nu) mice 5-6 weeks of age were implanted subcutaneously (s.c.) in their flanks with 2×10⁶ MCF-7 cells or 2×10⁶ NCFADR cells (obtained from the Tumor Repository Developmental Therapeutic Program, NCI—Frederick, Md.) suspended in Hank's balanced salt solution (HBSS). When the tumors exhibited a mean diameter of 7-8 mm, the mice were fed with M₄N in sterilized food balls (approx. 300 mg of M₄N in 2.5 ml of heated corn oil, mixed with 9 grams of Basal mix powder, Harlar Teklad Co., 2.0 ml of H₂O per ball). The mice were assigned to a treatment group that received M₄N dissolved in a 6% Cremaphor EL, 6% ethanol and 88% saline, and to a control group that received the vehicle only. Assignment was made so that both the control group and the experimental group contained mice bearing tumors of comparable sizes. Mice received a single daily 100 μL intraperitoneal (i.p.) injection containing 2 mg of M₄N for 3 weeks. The control mice received an equal volume of the vehicle. Tumors were measured in two perpendicular dimensions (L and W) once every seven days, and the tumor volumes were calculated according to the following formula: V=(L×W/2)³×π/6. The results from the individual mice were combined and the average tumor volume was determined. Statistical significance of the mean differences in tumor volume was assessed by Student's t-test.

### Results

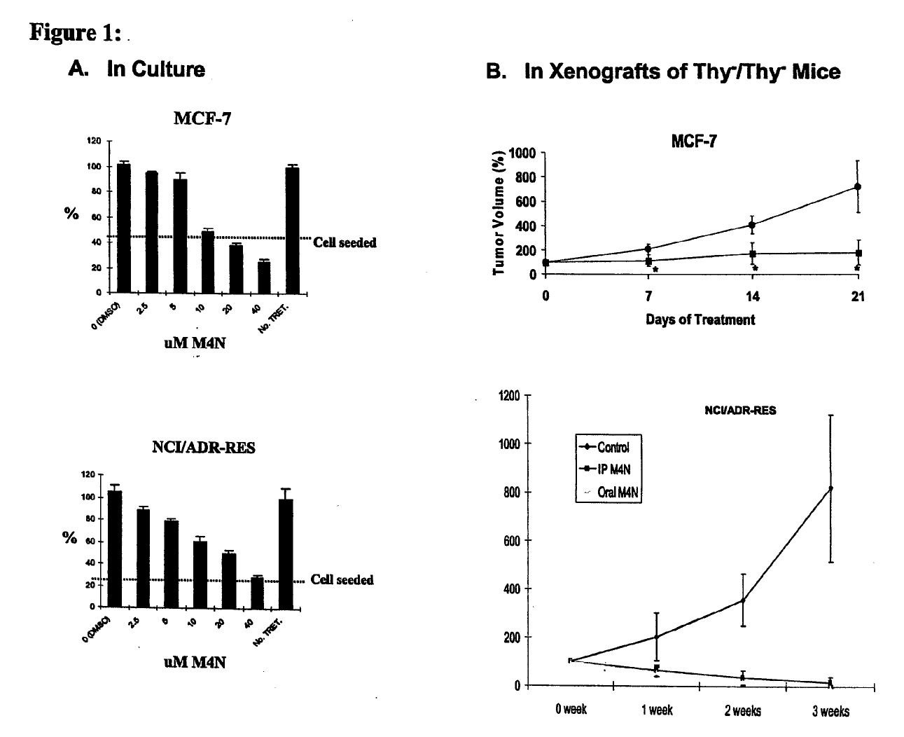

As shown in FIG. 1, M₄N was able to inhibit both MCF-7 and NCI/ADR-RES cellular growth both in culture (FIG. 1A) and in tumor xenografts (FIG. 1B). M₄N was more effective in inhibition of growth of MCF-7 than growth of NCI/ADR-RES. To achieve a total cell arrest in 72 hours, it required 40 μM of M₄N for NCI/ADR-RES while only 20 μM of M₄N was sufficient for MCF-7 cells. M₄N also greatly reduced the tumor sizes of both types of xenografts following 21 days of systemic treatments either by IP injection daily of M₄N or following oral feeding by adding M₄N in the food balls (FIG. 1B).

### Example 2

### Cdc2 and Survivin Expression are Greatly Reduced in MCF-7 and NCI/ADR Cells Following M₄N Treatment

### Methods

After treatment with indicated M₄N concentrations, MCF-7 and NCI/ADR cell monolayers were washed with PBS and harvested with 10 mM EDTA and 10 mM EGTA in PBS. The washed cells were pelleted and lysed in RIPA Buffer (50 mM tris-HCl, pH 7.4, 150 mM NaCl, 1% Triton x-100, 1% sodium deoxycholate, 0.1% SDS and 1 mM EDTA) containing Protease Inhibitor Cocktail (Sigma Chemical Co., St. Louis, Mo.). Protein concentrations were determined with the Bio-Rad protein concentration assay solution. Twenty-five micrograms of protein were separated on a 14% SDS PAGE gel and electroblotted to a Hybond enhanced chemiluminescence (ECL) nitrocellulose membrane (Amershamn Biosciences, Piscataway, N.J.) using a semi-dry electroblot apparatus. Primary rabbit polyclonal antibodies against Cdc2 (Oncogene Research Products, cat. #D04431-1), survivin (Santa Cruz Biotechnology, Santa Cruz, Calif., cat. #SCBT 10811), and cyclin B (Santa Cruz Biotechnology, cat. #SCBT H-433) and primary mouse polyclonal antibody actin (Santa Cruz Biotechnology, cat. #SC-8432) were used at a final concentration of 0.2 μg/mL. The secondary antibodies were anti-rabbit or anti-mouse IgGs conjugated to horseradish peroxidase. The filters were developed with the ECL Western Blot Detection Kit (Amersham). The chemiluminescence filters were placed against X-ray film for detection of protein bands.

### Results

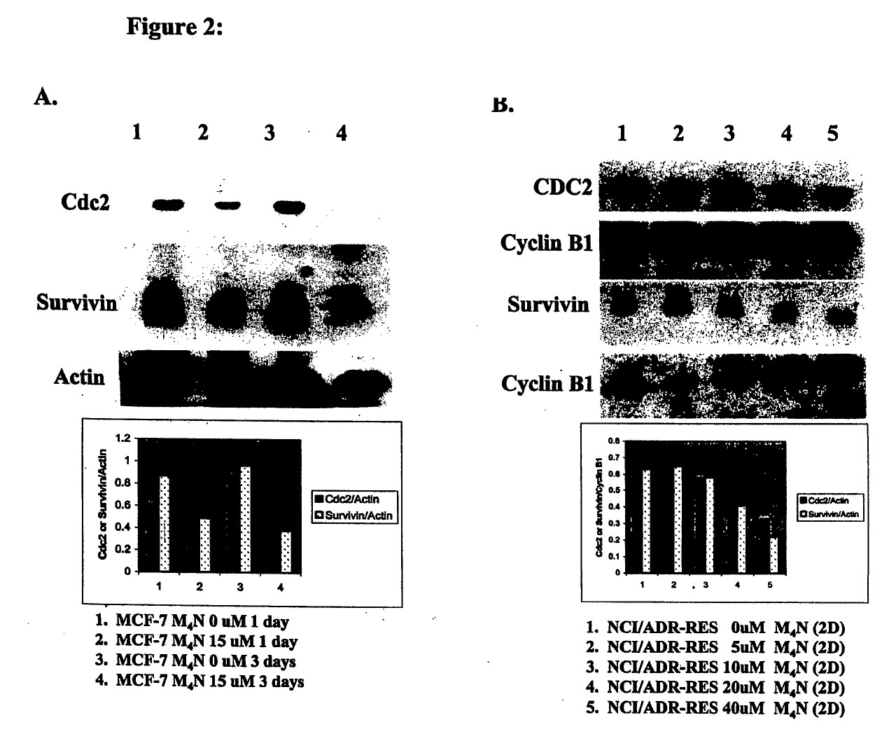

In addition to the effect on tumor growth (FIG. 1), M₄N treatment greatly inhibited Cdc2 and survivin gene expression in MCF-7 (FIG. 2A) and in NCI/ADR-RES cells (FIG. 2B). Upon 3 days of treatment of M₄N (15 μM), there was a >95% inhibition of Cdc2 and >60% of survivin in MCF-7 cells. Both Cdc2 and survivin in NCI/ADR-RES cells were also greatly reduced following 2 days of M₄N (40 μM) treatment (FIG. 2B).

### Example 3

### M₄N on Induction and Expression of MDR

### Methods

### Cell Culture and Drug Additions

The human breast cancer cell line, MCF-7, was obtained from ATCC. The multidrug resistant cell line, NCI/ADR-RES, was obtained from the DTP Human Tumor Cell Line Screen (Developmental Therapeutics Program, NCI). Both cell lines were maintained in Dulbecco's modified Eagle's medium supplemented with 10% fetal bovine serum and the antibiotics penicillin and streptomycin. The NDGA derivative, M₄N, was synthesized as described previously (1, 13). Stocks of M₄N and paclitaxel (Sigma-Aldrich, St. Louis, Mo.) were prepared in dimethyl sulfoxide (DMSO) and added to the cell culture medium so that the final concentration of DMSO was one percent. Aqueous stocks of Dox (Sigma-Aldrich) were prepared and filter sterilized before dilution into growth medium.

### Cytotoxicity Assay

NCI/ADR-RES cells were seeded at a density of 2×10⁴ cells per well in 24 well plates and 24 h later the growth medium was supplemented with M₄N, Dox, paclitaxel or combinations of M₄N with Dox or M₄N with paclitaxel. MAN was used at concentrations between 1.5 and 48 μM. For the combination of MAN and Dox a constant molar ratio of 2.4:1 (M₄N:Dox) was used and for M₄N and paclitaxel the ratio was 20:1 (M₄N:paclitaxel). Three days after drug addition cytoxicity was assessed with the SRB assay (14) with 540 and 690 nm (reference) absorbance measured with a Power Wave 200 microplate reader (Bio-Tek Instruments, Winooski, Vt.).

### Evaluation of Drug Interactions

The combination index (CD isobologram method of Chou and Talalay (16, 17, 18, 19) which is based on the median effect principle, was used to calculate synergism or antagonism for the combined drug effects. Dose-effect curves for each drug, singly and in combination, in serially-diluted concentrations were plotted using the median-effect equation and plot (18, 20) and the CI equation and plot (19). CI values at different effect and dose levels and isobolograms were generated automatically using the computer software CompuSyn (20). With this method, additive, synergistic or antagonistic effects are indicated by CI values of 1, <1, and >1, respectively. Comparison of the ratio of doses required to reach a given effect level for each single drug and the drugs in combination was used to determine the dose-reduction index (DRI).

### RT-PCR

Total RNA was isolated using the Trizol Reagent (Invitrogen, Carlsbad, Calif.). RT-PCR was carried out with the Superscript II One-Step RT-PCR system with Platinum Taq DNA Polymerase (Invitrogen) according to the manufacturer's protocol using oligonucleotide primers specific for MDR1:

| | |
|---|---|
| 5′-ACATGACCAGGTATGCCTAT-3′ | (SEQ ID NO:1) |
| 5′-GAAGATAGTATCTTTGCCCA-3′ | (SEQ ID NO:2) |

### and GAPDH:

| | |
|---|---|
| 5′-CCATCACCATCTTCCAGGAG-3′ | (SEQ ID NO:3) |
| 5′-CCTGCTTCACCACCTTCTTG-3′ | (SEQ ID NO:4) |

The RT-PCR products were separated by agarose gel electrophoresis and stained with ethidium bromide. Relative band intensities were quantified by ImageJ software (NIH, Bethesda, Md.).

### Western Blotting

Cultured cells were incubated in a solution of 10 mM EDTA and 10 mM EGTA in PBS and then harvested by scraping with a Teflon® cell scraper. The cells were pelleted and lysed in modified RIPA buffer [50 mM Tris-HCl (pH 7.4), 1% NP-40, 0.25% Na-deoxycholate, 150 mM NaCl, 1 mM EDTA (pH 8.0)] containing Protease Inhibitor Cocktail (Sigma Chemical Co.). The lysate was cleared by centrifligation and protein concentrations were determined with the Bio-Rad Protein Assay (Bio-Rad Laboratories, Hercules, Calif.). Protein samples were separated by SDS-PAGE and electroblofted to a Hybond enhanced chemiluminescence (ECL) nitrocellulose membrane (Amersham) with a semidry blotting apparatus and detected as described in the instruction manual of the ECL Western Blotting System (Amersham). The antibodies used were primary rabbit polyclonal antibodies against Pgp and cyclin B and a horseradish peroxide conjugated secondary antibody (Santa Cruz Biotechnology). Relative band intensities were measured with ImageJ software.

### Rhodamine 123 Efflux Assay

NCI/ADR-RES cells were cultured in the presence of 0, 1.25, 2.5, 3.75 and 5.0 μM M₄N. After three days the cells were harvested by trypsinization and resuspended at a density of 5.0×10⁶ cells/ml in the same media containing 1.0 μg/ml Rhodamine 123 (Sigma-Aldrich, St. Louis, Mo.). Following a one hour incubation at 37° C., the Rhodamine loaded cells were washed twice with ice-cold PBS and resuspended in media with the starting concentrations M₄N. During the efflux phase, 5.0×10⁵ cells were collected every 15 minutes, washed in ice-cold PBS, resuspended in 100 μl of ice-cold PBS and analyzed by fluorometry at an excitation wavelength of 485 nm and an emission wavelength of 535 nm.

### Results

### Evaluation of the Combined Effect of M₄N and the Chemotherapeutic Agents Dox and Paclitaxel

M₄N, Dox, and paclitaxel as single agents or in combination inhibited the growth of the multidrug resistant human breast cancer cell line NCFADR-RES in a dose dependent manner. The average IC₅₀ value for M₄N was 8.67 μM, while the IC₅₀ values for Dox and paclitaxel were 3.22 μM and 3.26 μM respectively (Table 1). The IC₅₀ values for Dox and paclitaxel are indicative of the MDR phenotype displayed by NCI/ADR-RES cells as the reported IC₅₀ values for these two agents are 130 nM and 6.4 nM for MCF-7, a drug sensitive human breast cancer cell line (10). Conversely, the IC₅₀ value for M₄N against MCF-7 from our previous studies (5) is approximately 7 μM, nearly the same as that for the drug resistant cell line. When used in combination in NCI/ADR-RES cells, M₄N and Dox had IC₅₀ values of 5.63+2.34 μM. For the M₄N and paclitaxel combination the IC₅₀ values were 3.31+0.17 μM (Table 1).

TABLE 1
Dose-effect relationships of M₄N, alone and in combination with doxorubicin
or paclitaxel, in human multidrug resistant breast cancer cells.

| | Parameters ^{a} | | | CI ^{b} value at | | | | DRI ^{c} value at | | | |
|---|---|---|---|---|---|---|---|---|---|---|---|
| Drugs | Dₘ | m | r | ED₅₀ | ED₇₅ | ED₉₀ | ED₉₅ | ED₅₀ | ED₇₅ | ED₉₀ | ED₉₅ |
| M₄N | 8.19 | 0.52 | 0.92 | | | | | 1.46 | 5.94 | 24.26 | 63.16 |
| Dox | 3.22 | 0.49 | 0.99 | | | | | 1.37 | 6.47 | 30.50 | 87.56 |
| M₄N/Dx | 5.63 + 2.34 | 1.57 | 0.99 | 1.42 | 0.32 | 0.07 | 0.03 | | | | |
| M₄N | 9.14 | 0.91 | 1.00 | | | | | 2.75 | 3.98 | 5.76 | 7.40 |
| Pctxl | 3.26 | 0.59 | 0.97 | | | | | 19.63 | 54.32 | 150.31 | 300.34 |
| M₄N/Px | 3.31 + 0.17 | 1.32 | 0.98 | 0.41 | 0.27 | 0.18 | 0.14 | | | | |

^{a} Dₘ, median effect dose (concentration in micromoles/liter that inhibits cell growth by 50%).
m, shape of the dose-effect curve (m = 1, hyperbolic; m > 1, sigmoidal; m < 1, negative sigmoidal).
R, linear correlation coefficient of the median effect plot.
^{b} CI, combination index (CI < 1, synergism; CI = 1, additive effect; CI > 1, antagonism).
^{c} DRI, dose reduction index (measured by comparing the doses required to reach a given degree of inhibition when using the drug as single agent and in combination.

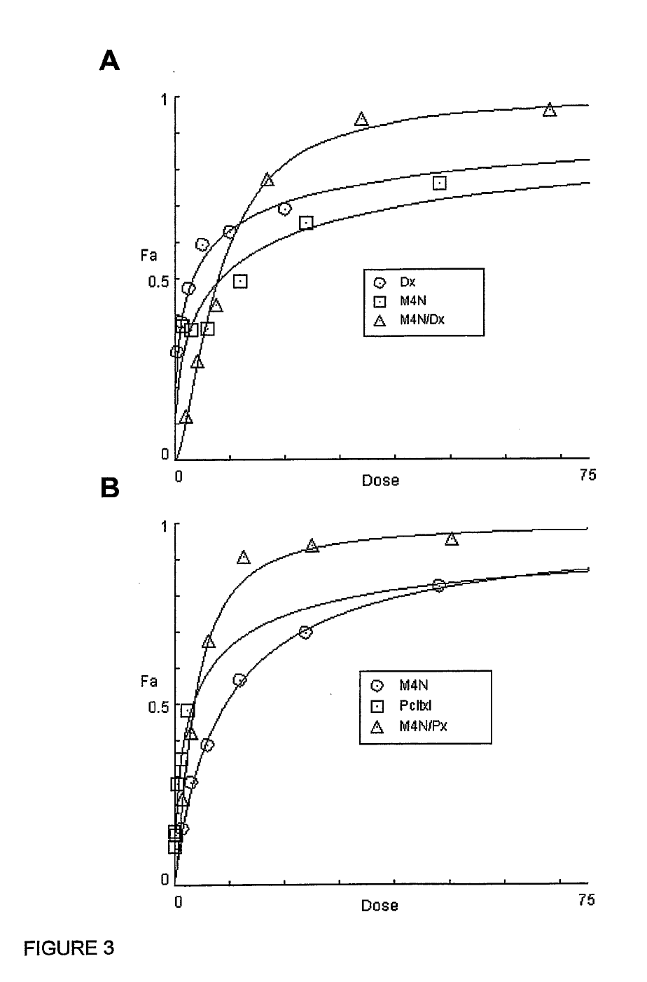

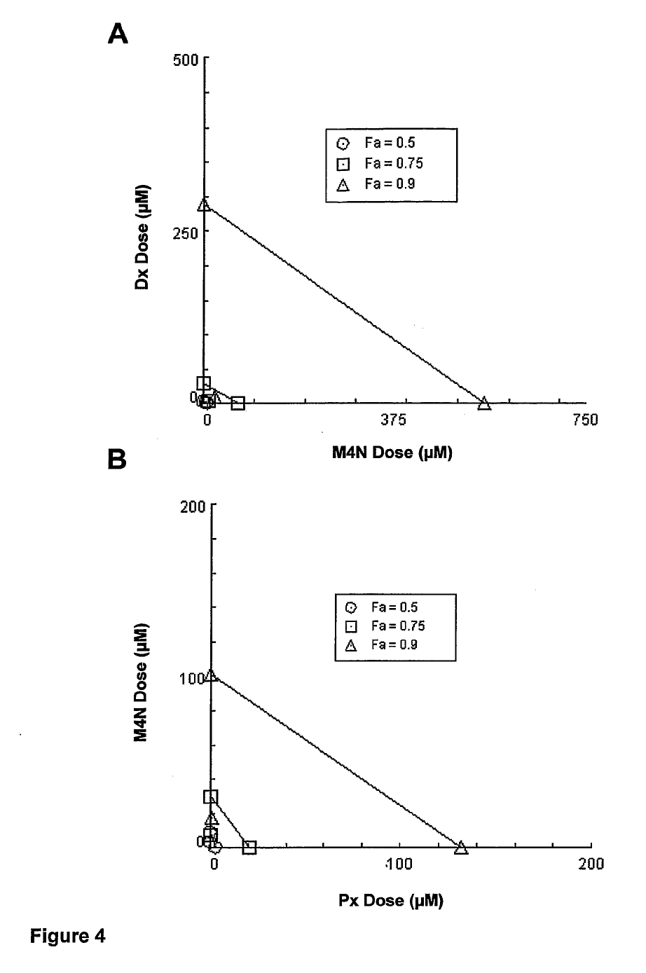

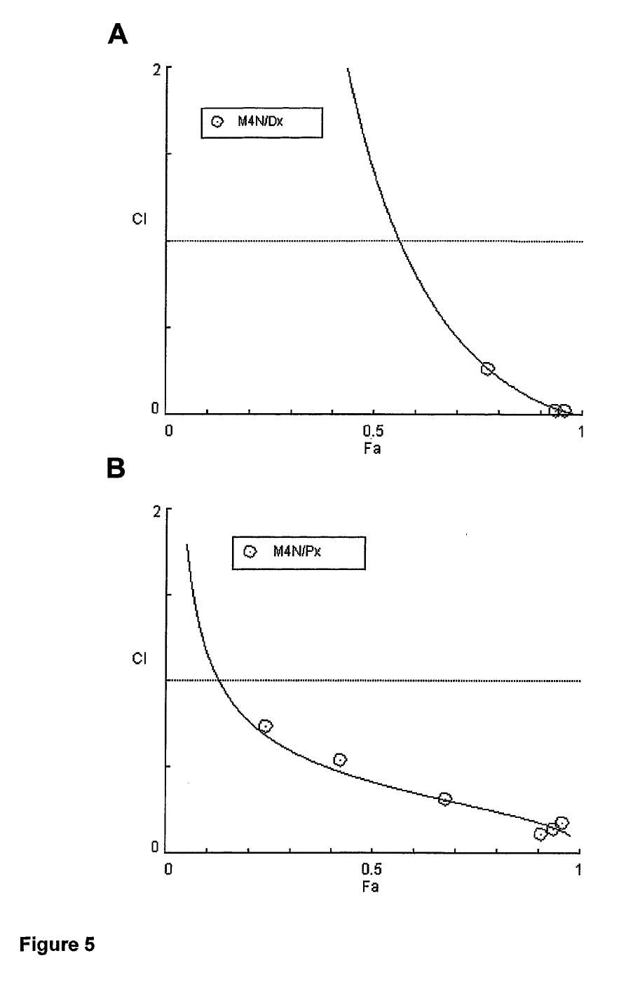

Two methods, the isobologram method and the combination index (CI) method, were used to determine if there is synergy between M₄N and Dox or paclitaxel. Isobolograms were constructed for the doses of M₄N and Dox and M₄N and paclitaxel necessary to inhibit growth 90% (Fa=0.9), 75% (Fa=0.75) and 50% (Fa=0.5). The experimental data points for both drug combinations were at drug concentrations below the expected additive effect line for each of these values, indicating that there is a strong synergy (i.e., CI<0.3) between M₄N and the two secondary chemotherapeutic agents (FIG. 3). In addition, the median effect analysis of Chou and Talalay (19) was used to calculate the combination index (CI) for the two drug combinations. The M₄N and Dox combination was very strongly synergistic (CI<0.1) at high dose levels (ED₉₀ and ED₉₅), whereas M₄N and paclitaxel were strongly synergistic (CI=0.41-0.14) across the entire range of doses (FIGS. 3-5, Table 1).

The dose reduction index (DRI) determines the fold dose-reduction allowed for each drug in synergistic combinations. This is important since dose reduction results in reduced toxicity while maintaining the desired efficacy. As a result of their synergism, the DRI exhibited a sizeable dose reduction for each of the drugs (Table 1). The DRI indicated that the concentration of Dox necessary to inhibit the growth of 75% of NCI/ADR-RES cells (ED₇₅) could be decreased 6.47 fold, i.e. from 30.5 μM to 4.7 μM by the concurrent administration of 11.3 μM M₄N, and the ED₉₅ could be reduced 87.6 fold. Similarly, the ED₅₀ of paclitaxel could be decreased 19.63 fold and the ED₉₅, 300.3 fold.

TABLE 2
Effect of M₄N, Maltose-M₃N and Paclitaxel, Alone and in
Combination, on the Growth of NCI/ADR-RES Xenografts

| | | | Mean Body | | | | |
|---|---|---|---|---|---|---|---|
| | | | Weight | | | | |
| | Dosage | | Change | | | Relative Mean | |
| Group | (μmoles/m²) | Route | (g/mouse) | Deaths | Tumors | Tumor Volume | T/C % |
| Control | 0 | i.p. | 0.0 | 0/7 | 14 | 2.62 | |
| M₄N | 320 | i.p. | −0.2 | 0/7 | 14 | 1.33 | 50.8 |
| Px | 16 | i.p. | −0.9 | 0/7 | 11 | 1.59 | 60.7 |
| M₄N + Px | 320 + 16 | i.p. | +0.3 | 0/7 | 8 | 0.94 | 35.9 |
| M₄N | 160 | i.p. | +0.2 | 0/7 | 13 | 1.70 | 64.9 |
| Px | 8 | i.p. | +0.3 | 0/7 | 12 | 0.90 | 34.4 |
| M₄N + Px | 160 + 8 | i.p. | +1.3 | 0/7 | 11 | 0.82 | 31.3 |
| Mal-M₃N | 320 | i.p. | +0.6 | 0/7 | 13 | 1.25 | 47.7 |
| Mal-M₃N + Px | 320 + 16 | i.p. | +0.2 | 0/7 | 14 | 0.65 | 24.8 |

### Example 4

### M₄N Inhibition of MDR1 Gene Expression and P-Glycoprotein Levels

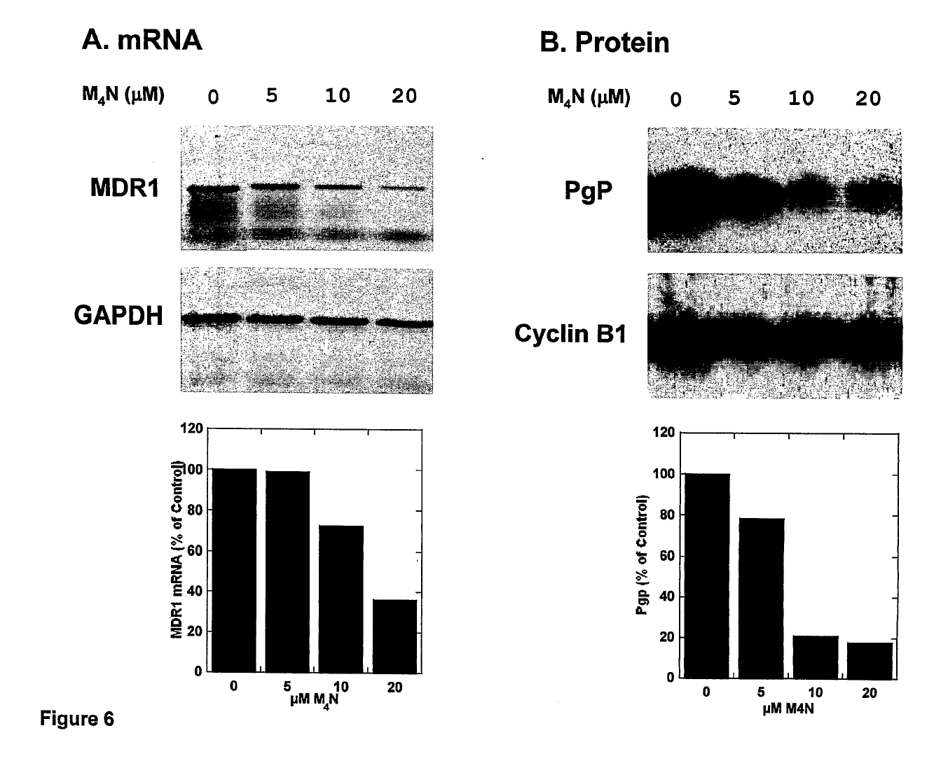

We next examined the effect of M₄N on MDR1 gene expression to determine whether the observed synergy between M₄N and Dox and paclitaxel in multidrug resistant cells is the result of reversal of the MDR phenotype. We postulated that M₄N, through its ability to inhibit Sp1 binding, might down regulate MDR1 gene expression. To examine this possibility, NCI/ADR-RES cells were exposed to 0, 5, 10 and 20 μM M₄N for three days, after which total RNA and protein were examined for levels of MDR1 mRNA and Pgp. After treatment with 20 μM M₄N, the level of MDR1 MRNA in the cells was reduced to 36.3% of the untreated value after normalization to the housekeeping gene GAPDH (FIG. 6A). The amount of Pgp was also reduced with its abundance decreasing to 17.8% of the control amount after a three day exposure to 20 μM M₄N (FIG. 6B). Even a three day exposure to 5 μM M₄N resulted in a 21.8% reduction in Pgp. The levels of Pgp were normalized to cyclin B1, whose expression, according to our previous results, is unaffected by M₄N (4, 5).

The results indicate that M₄N may be able to reverse the MDR phenotype by inhibiting the constitutive expression of MDR1 mRNA and Pgp in multidrug resistant cells.

### Example 5

### Effect of M₄N on Ability of Cells to Acquire Resistance after Chemotherapy

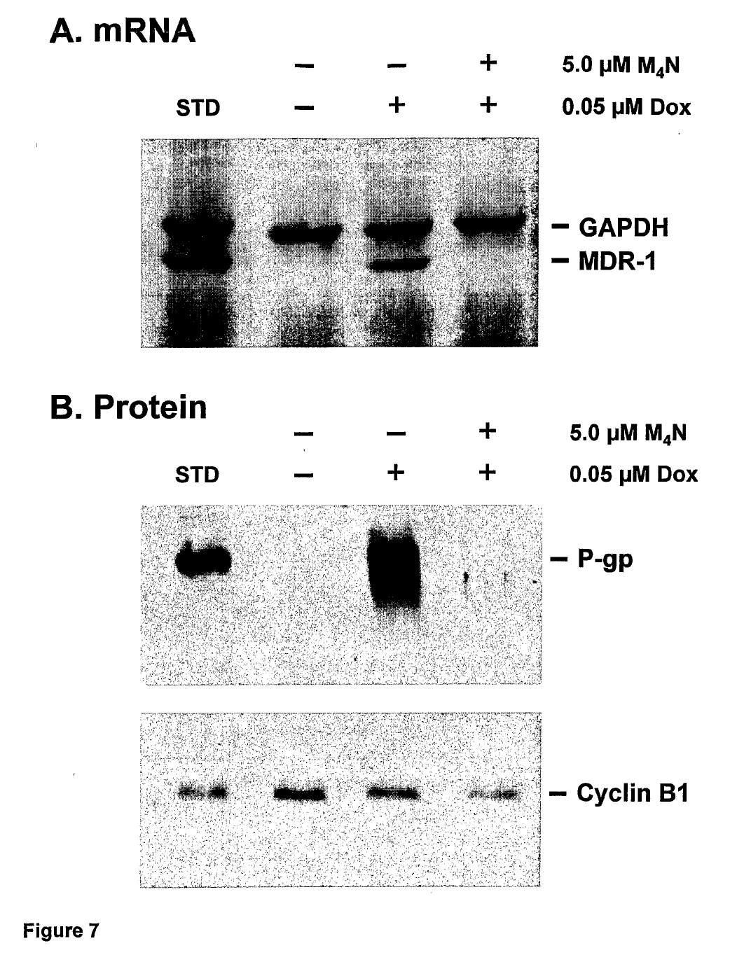

Next we investigated whether M₄N could be used to prevent cells from acquiring resistance after exposure to chemotherapy. MDR1 gene expression is induced when the drug sensitive human breast cancer cell line is exposed to low doses of Dox. We treated MCF-7 cells for two days with 0.05 μM Dox in the presence or absence of 5.0 μM M₄N and measured the relative amounts of MDR1 mRNA and Pgp protein. Treatment with Dox in the absence of M₄N induced measurable expression of both MDR1 mRNA and Pgp (FIG. 7). MDR1 expression was not detectable in MCF-7 cells without exposure to Dox. Induction of MDR1 expression was abolished, however, by combination treatment with M₄N (FIG. 7).

### Example 6

### Effect of M₄N on Rhodamine-123 Efflux in Multidrug Resistant Cells

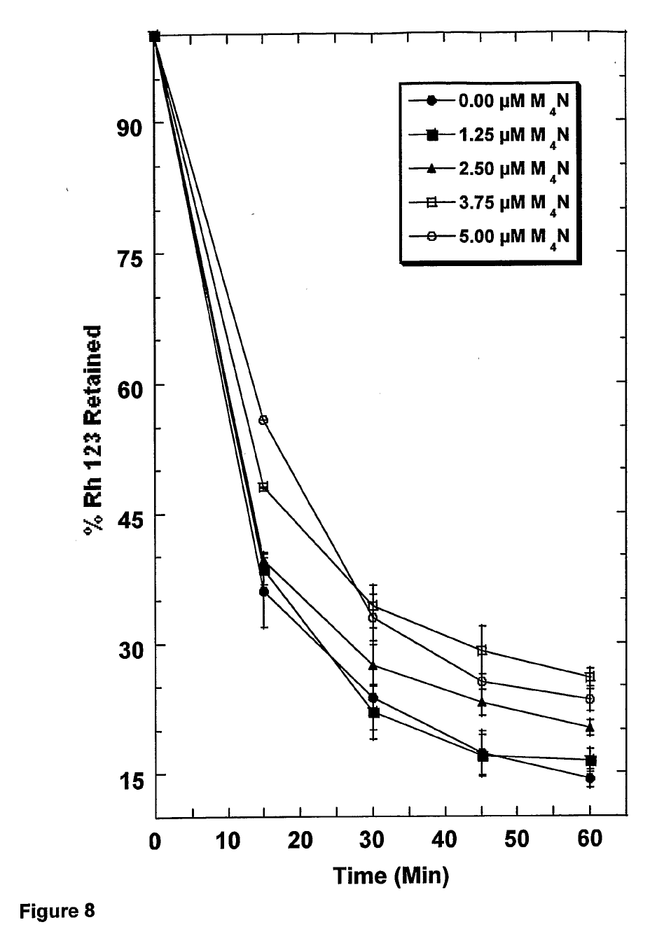

Efflux of Dox and paclitaxel from multidrug resistant cells is mediated by Pgp. The Pgp substrate Rh-123 was used to examine the effect of Pgp down regulation by M₄N on drug efflux. NCI/ADR-RES cells were incubated for three days in the presence of 0, 1.25, 2.5, 3.75 and 5.0 μM M₄N. The cells were then loaded with Rh-123, washed and allowed to efflux with or without M₄N. During the efflux period, the cells were assayed for the amount of cell-associated Rh-123 at 15 min intervals for an hour. Untreated resistant cells had an E₅₀ (time at which 50% of Rh-123 is retained by the cells) of approximately 12 minutes (FIG. 8). Treatment of cells with 1.25, 2.5, 3.75 and 5.0 μM M₄N increased the E₅₀ to 12.5, 12.5, 15 and 20 minutes respectively. These results on slowing down the drug efflux are consistent with the reduction of Pgp levels in cells by M₄N.

### Example 7

### Saccharide Substituted M₄N Derivatives

We synthesized a novel water soluble M₄N analogue, Maltose-M₃N, by replacing a methyl group on M₄N at 3′ or 4′ position with a maltose molecule. In vitro and in vivo anticancer activity of Maltose-M₃N against five human cancer cell lines was evaluated. Its inhibitory effect on Cdc2 and survivin expression was also examined.

### Methods

### Synthesis of Maltose-M₃N

M₃N was obtained as a by-product from the synthesis of M₄N, and purified with silica gel chromatography (1). M₃N (0.47 g) and β-maltose octa-acetate (1.85 g) (21, 23) were dissolved in dichloromethane (5 ml), and treated with boron trifluoride etherate (2.0 ml) for 3-4 hours. After decomposition of excess boron trifluoride, the organic solution was washed with cold solutions of sodium bicarbonate and sodium chloride, evaporated to a syrup, and dissolved in 95% EtOH for chromatography in a column of Sephadex LH-20 (5×200 cm). The product was separated by chromatography from the excess maltose acetate as well as unreacted M₃N (the unreacted M₃N can be reused for another round of maltosylation). Maltosylated M₃N (solid) is deacetylated in dry methanol with catalytic amount of sodium methoxide, and sodium methoxide is removed with Dowex 50×8 (hydrogen form). Evaporation of the methanolic solution yielded solid product. The final isolated yield was 35-40% of the starting M₃N, but the yield may be increased to 60% or greater by reglycosylating the unreacted M₃N.

Galactose M₃N and other saccharide derivatives may be made using this method with appropriate changes to the starting compounds, as will be appreciated by those of skill in the art. Such compounds can be tested for efficacy using the methods described above and below.

### Cell Culture and Maltose-M₃N Treatment

Human tumor cell lines were obtained from ATCC (Mannassas, Va.). The human hepatocellular carcinoma cell line, Hep3B, was maintained in Eagle's MEM supplemented with 10% FBS; the human breast cancer cell line, MCF7, was grown in DMEM containing 10% FBS; the human colorectal carcinoma cell line, HT29, was cultured in McCoy's 5a medium with 10% FBS; the human prostate carcinoma cell line, LNCaP, was maintained in RPMI 1640 with 10% FBS and human erythroleukemia cell line, K562, was propagated in Iscove's modified Dulbecco's medium (IMDM) containing 10% FBS. All of the cultures contained the antibiotics penicillin and streptomycin. The C3 cell line was generated by transfection of the EJras-transformed C57B16 (B6) mouse embryo cells with fall length HPV16. C3 cells were grown and maintained in IMDM supplemented with 5% FBS plus penicillin and streptomycin.

For cell culture experiments, Maltose-M₃N was dissolved in water to a concentration of 10 mM and then sterilized by filtration. Cells were seeded at approximately 2×10³ cells per cm² in complete media. Twenty-four hours after seeding, the growth media was removed and replaced with media containing the desired concentrations of Maltose-M₃N.

### Cell Viability

Twenty thousand cells were seeded into each well of 24-well plates. Twenty-four hours later, the medium was replaced with new medium containing various concentrations of Maltose-M₃N. After 3 days of incubation, the medium was changed to MTT solution containing 500 μg MTT, 5% FBS, and 100 units/ml of penicillin and streptomycin dissolved in PBS. Two hours after incubation, MTT solution was removed and replaced with 500 μl DMSO/well. The solubilized dye was transferred to 96-well plates and read at the wavelength of 540 nm using an ELISA reader.

### Western Blotting

After the desired drug incubation times, media were collected and monolayer cell cultures were scraped with a Teflon® policeman in a solution of 10 mM EDTA and 10 mM EGTA in PBS. The cells were pelleted and lysed in modified RIPA buffer [50 mM Tris-HCl (pH 7.4), 1% NP-40, 0.25% Na-deoxycholate, 150 mM NaCl, 1 mM EDTA (pH 8.0), and lx Protease Inhibitor Cocktail (Sigma Chemical Co.)]. Protein extracts were then quantified with the Bio-Radprotein concentration assay solution and stored at −80° C. For western blots, proteins were separated by SDS-PAGE and electroblotted to a Hybond enhanced chemiluminescence nitrocellulose membrane (Amersham) with a semidry blotting apparatus and detected as described in the instruction manual of the enhanced chemiluminescence kit (Amersham Pharmacia). The antibodies used were the primary rabbit polyclonal antibodies against Cdc2, survivin, and β-actin and the horseradish peroxide-secondary antibody (Santa Cruz Biotechnology).

### Maltose-M₃N Treatment of C3 Cell-Induced Tumors in Mice

Four C57b1/6 mice were inoculated with 5×10⁵ C3 cells subcutaneously between the shoulders. Tumors were allowed to develop for approximately 20 days. The mice then received daily intratumoral injections of 0.1 ml of 0.15 M NaCl, 10 mg, 20 mg or 40 mg of Maltose-M₃N dissolved in 0.15 M NaCl. After 4 days of treatment, the mice were euthanized and the tumors were excised, immediately fixed and then stored in 4% formaldehyde in PBS. Tissue samples were then sent to Paragon Bioservices (Baltimore, Md.) for histology and immunohistochemistry using antibodies against Cdc2 and survivin.

### Results

### In Vitro and In Vivo Anticancer Activity of Maltose-M₃N Against Human Tumors

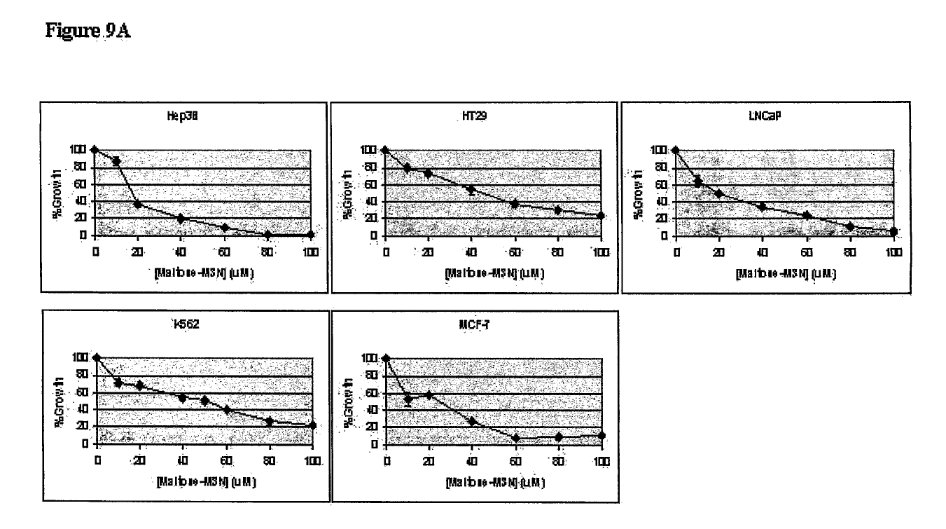

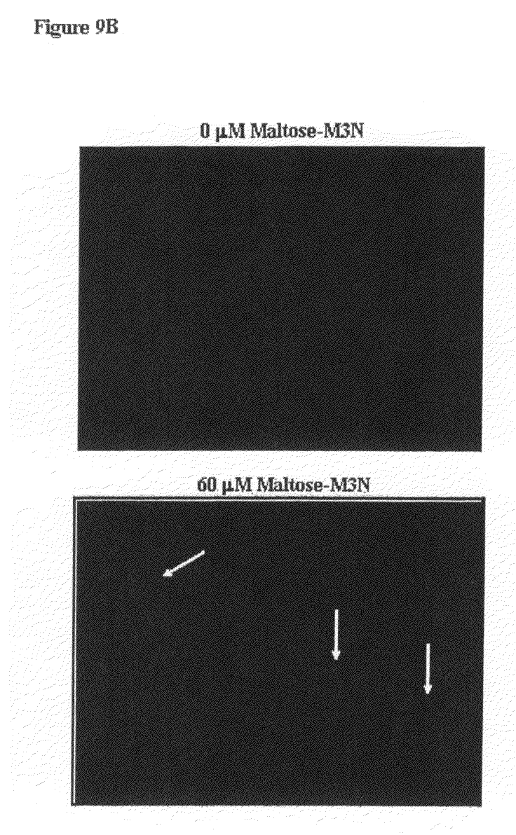

The growth inhibitory effect of Maltose-M₃N was evaluated in five human cancer cell lines, including Hep3B, HT29, K562, LNCaP, and MCF7. Exposure of the cells to Maltose-M₃N for 3 days resulted in a dose-dependent decrease in cell viability in every cell line tested with IC₅₀ values between 20 μM and 40 μM, very similar to the patterns observed for M₄N (FIG. 9A). DAPI staining of the nuclei of Maltose-M₃N-treated Hep3B was performed to identify condensed nuclei of apoptotic bodies (FIG. 9B) and confirmed the Maltose-M₃N associated cell death occurred via apoptosis.

### Inhibition of Cdc2 and Survivin Gene Expression by Maltose-M₃N

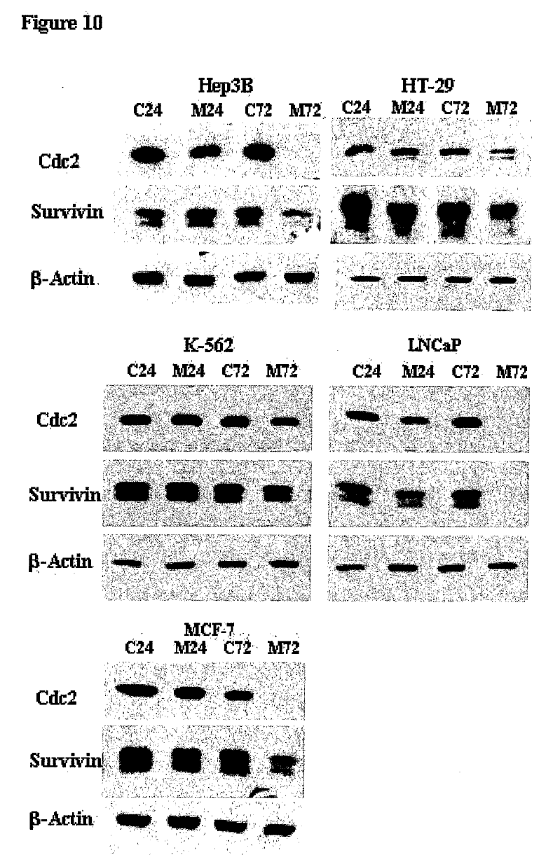

To examine whether Maltose-M₃N suppresses Cdc2 and survivin protein productivity, cell cultures were exposed to a growth inhibitory dose of Maltose-M₃N and protein was extracted after 24 and 72 hours of treatment. Western blot analysis of the protein samples showed decreased Cdc2 and survivin protein expression in the treated cells. Compared to MEN, Maltose-M₃N exhibited similar inhibition of Cdc2 and survivin protein expression (FIG. 10).

### In Vivo Antitumor Activity

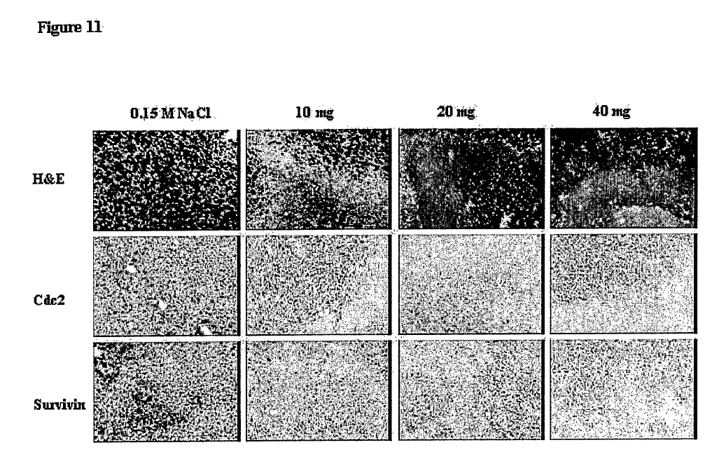

In a preliminary test for in vivo antitumor efficacy of Maltose-M₃N, C3 tumors received single daily intratumoral injections containing various concentrations of Maltose-M₃N for 4 days. Following the treatments, the mice were euthanized, photographed, and tissue samples were analyzed by H&E, Cdc2, and survivin staining. The color of the treated tumors appeared darker compared to that of the control. The color change was associated with elevated levels of apoptosis and necrosis as evidenced by the H&E staining. In addition, the treated tumors clearly showed reduced expression levels of both Cdc2 and survivin protein compared with the control tumor (FIG. 11).

### Example 8

### Combination Therapy of M₄N or Maltose-M₃N and Paclitaxel Against NCI/ADR-RES Resistant Breast Cancer Xenografts in Nude Mice

### Methods

Nude mice bearing NCI/ADR-RES multidrug resistant breast cancer xenografts were used as a model for combination therapy to further examine synergy between M₄N and paclitaxel.

T-cell deficient female nude (nu/nu) mice, 5-6 weeks of age, were purchased from Charles River Laboratories (Wilmington, Mass.) and were housed in a pathogen-free room. All experiments involving the mice were carried out in accordance with the Johns Hopkins University Animal Care and Use Committee guideline. The mice were implanted subcutaneously in both flanks with 1×10⁶ NCI/ADR-RES cells suspended in Hank's balanced salt solution (HBSS). When the tumors exhibited a mean diameter of 2-4 mm, the mice were randomly assigned to treatment groups (7 mice per group), solvent alone, that received M₄N or Maltose-M₃N alone, or in combination with paclitaxel. For intraperitoneal (i.p.) administration, M₄N, maltose-M₃N and paclitaxel were dissolved in a recently developed reduced cremophor solution containing 20% (v) dehydrated ethanol, 20% (v) Cremophor EL, PEG 300, <11% (v) Tween 80 (22). Daily injections of 0.05 ml were performed for each drug and drug combination.

Tumors were measured in two perpendicular dimensions once every seven days, and the tumor volumes were calculated according to the following formula:

Tumor volume=(a²×b)/2

where a is the width of the tumor (smaller diameter) and b is the length (larger diameter) (23). The mean tumor volume and standard error were calculated for each treatment group. Relative mean tumor volumes, (V/V₀), where V is the mean tumor volume at a particular time and V₀ is the mean tumor volume at day 0 (23), were determined and used to assess tumor growth inhibition (T/C value) using the following equation:

T/C(%)=Relative Mean Tumor Volume of treated/Relative Mean Volume of control×100.

The NCI standard for the minimal level for antitumor activity (T/C≦42%) was adopted (25). At the termination of the experiment the tumors were excised and fixed in formaldehyde. Tissue samples were then sent to Paragon Bioservices for histology and immunohistochemistry using antibodies against human P-glycoprotein (Pgp).

Two dosage regimens of M₄N and paclitaxel, both with a constant molar ratio of 20:1 (M₄N:paclitaxel), were employed as determined by in vitro testing to be synergistic as shown in Table 1. The 8 and 16 μmol m² doses of pactitaxel were submaximal based on values from other studies (22) and the M₄N doses of 160 and 320 μmol/m² were arrived at by decreasing the maximum tolerated dose used in our previous study with MCF-7 breast cancer xenografts (5). An additional NDGA derivative was also tested. Maltose-M₃N was developed as a water soluble alternative to M₄N, however for consistency it was dissolved in the same reduced cremophor solvent system as M₄N and paclitaxel for this study. For control mice receiving the solvent only, the explanted tumors increased appreciably over two weeks of treatment, with the mean tumor volume nearly tripling in size (Table 2). Tumor growth was also noted in mice treated with submaximal doses of either M₄N (320 μmol m²), paclitaxel (16 μmol m²) or maltose-M₃N (320 μmol/m²) with relative mean tumor volumes after two weeks of 1.33, 1.59 and 1.25 respectively. For the mice treated with a combination of M₄N and paclitaxel or maltose-M₃N and paclitaxel, however, the final relative mean tumor volumes were less than one, indicating an overall decrease in tumor size (Table 2). Moreover the tumor growth inhibition (T/C) values for each of the drug combination regimens were all lower than ≦42%, the minimum level for antitumor activity according to National Cancer Institute standards (24).

The health and well being of the mice were assessed by recording their body weights at the beginning and end of the treatment period. For each of the dosage regimens the mean change in body weight was small (−0.9 to +1.3) and not significantly different than that for the control group (Table 2). The only two treatment groups exhibiting a decrease in mean body weight were the higher dose single drug regimens of M₄N and paclitaxel (−0.2 and −0.9 respectively). There were no mouse deaths recorded in any of the groups. An advantage of combination therapy with synergistic drugs is the ability to use submaximal doses of the chemotherapeutic agents. This is reflected in the decreased toxicity of the treatment regimens as illustrated by the stability of body weight and the zero mortality rate.

### Example 9

### M₄N and Maltose-M₃N Inhibition of P-Glycoprotein Levels in NCI/ADR-RES Breast Cancer Xenograft Tumors

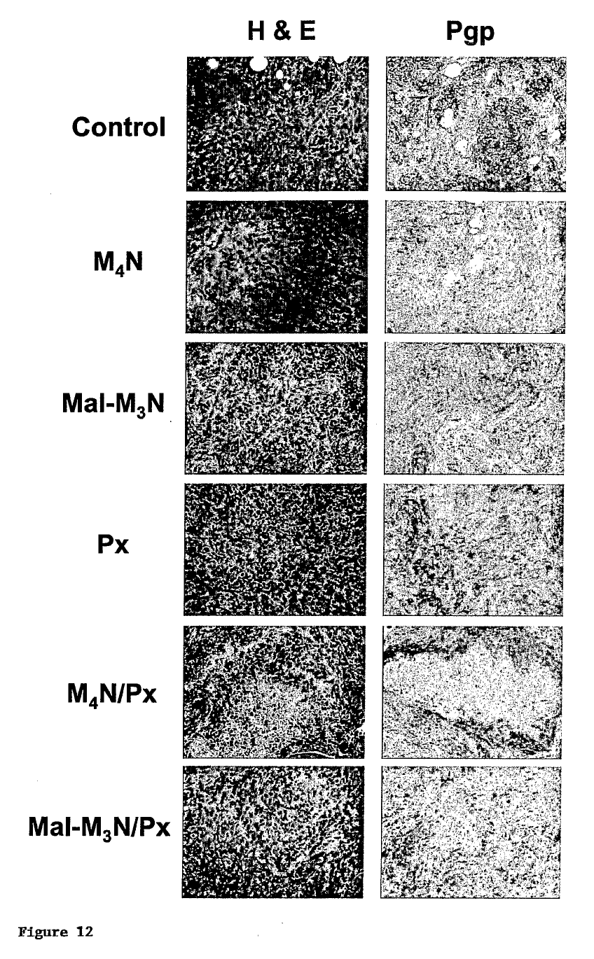

In order to examine the mechanism underlying the apparent synergism between M₄N and paclitaxel in inhibiting the growth of the drug resistant breast cancer xenografts, the tumors were analyzed for the presence of Pgp. Tissue sections from xenograft tumor biopsies from each of the treatment groups in Example 8 were either stained with hematoxylin and eosin (H&E) or immunochemically using Pgp specific antibodies. Tumor sections from the control group exhibited robust antibody brown color staining for Pgp at the surface of most of the tumor cells (FIG. 12). A similar pattern was seen in the paclitaxel treated tumors. In contrast the M₄N and maltose-M₃N treated tumors showed a dramatic decrease in antibody staining consistent with a decrease in the amount of Pgp protein. Tumors treated with a combination of M₄N and paclitaxel should result in tumor regression with some central necrosis by H&E staining and an absence of Pgp antibody staining (FIG. 12). Pgp levels were similarly decreased in the maltose-M₃N/Paclitaxel treated tumors.

### Example 10

### NDGA Derivatives Containing Longer Chain Amino Acids

NDGA derivatives wherein R₁-R₄ comprise at least one “long chain” amino acid substituent, and derivatives thereof (as defined hereinabove), are also expected to be useful. Such substituents have at least two —CH₂— groups (generally 2-4) present between the amino and carboxyl groups. Derivatives of these compounds include, inter alia, compounds wherein the amine group has a dimethyl substitution, for example:

5-((2S,3R)-4-{3,4-bis[4-(dimethylamino)butanoyloxy]phenyl}-2,3-dimethylbutyl)-2-[4-(dimethylamino)butanoyloxy]phenyl 4-(dimethylamnino)butanoate

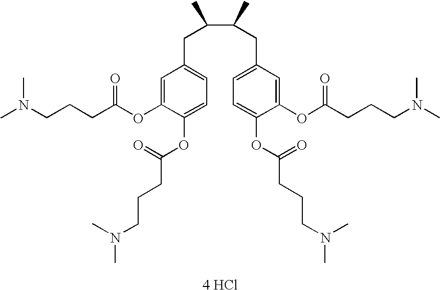

5-((2S,3R)-4-{3,4-bis[4-(dimethylamino)propanoyloxy]phenyl}-2,3-dimethylbutyl)-2-[4-(dimethylamino)propanoyloxy]phenyl 4-(dimethylamino)propanoate

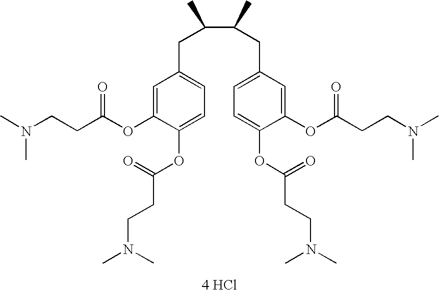

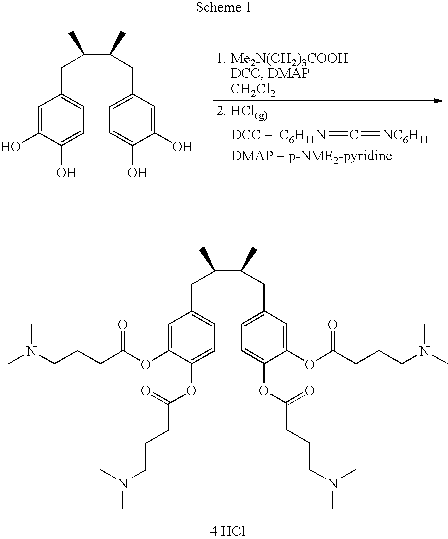

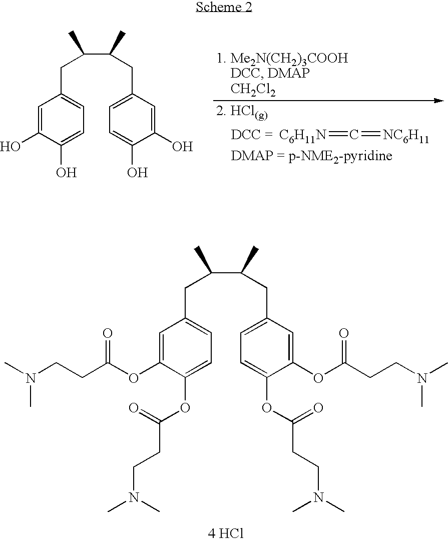

Such derivatives can be made by those of skill in the art by routine methods. For example, the following procedure can be used, with appropriate substitution of starting material to obtain other similar derivatives.

5-((2S,3R)-4-{3,4-bis[4-(dimethylamino)butanoyloxy]phenyl}-2,3-dimethylbutyl)-2-[4-(dimethylamino)butanoyloxy]phenyl 4-(dimethylamino)butanoate (Meso-2,3-dimethyl-1,4-bis{3,4-bis)[4-(dimethylamino)butanoyloxy]phenyl}butane) (see Scheme 1)

To a solution of NDGA (1.05 g, 3.46 mmol, 1.0 equiv) and 4-(dimethylamino)butyric acid hydrochloride (3.48 g, 20.76 mmol, 6.0 equiv) in dichlorormethane (100 mL) was added DCC (4.28 g, 20.76 mmol, 6.0 equiv) and DMAP (422.7 mg, 3.46 mmol, 1.0 equiv). The reaction mixture was stirred under nitrogen at room temperature for 24 h. After dicyclohexylurea in the reaction mixture was filtered off, the resultant solution was concentrated under reduced pressure. Acetone (250 mL) was then added into the residue and the resultant solution was bubbled with excel HCl (g). The precipitate was dissolved in water and re-precipitated twice by use of acetone at room temperature to give the product as white solids.

Much attention and resources have been directed toward reversing the resistance to multiple anticancer drugs that can develop after courses of adjuvant chemotherapy. First generation MDR reversal agents were pharmacologically active compounds that also happened to bind Pgp. These drugs were ultimately unsuccessful because their other pharmacological properties made them too toxic for clinical use. The present inventors have shown that M₄N and other NDGA derivatives can reverse the MDR phenotype in multidrug resistant cancer cells. M₄N and other NDGA derivatives are uniquely suited to perform the task of resensitizing cells to chemotherapeutic drugs such as Dox and paclitaxel. Furthermore, the compounds of the invention are also able to inhibit Dox-mediated induction of MDR1 gene expression, and should therefore be useful in preventing the development of MDR if administered during the initial stages of chemotherapy. These findings result in several useful strategies for the treatment of cancer. For example, patients whose cancers have become resistant to multiple anticancer agents can be treated with the compounds of the invention to reverse the MDR phenotype of the cancer cells, followed by retreatment with the original chemotherapeutic agents or others (e.g. Dox or palictaxil). Furthermore, the compounds of the invention can be added in low doses to the initial adjuvant chemotherapy regimen to prevent the development of MDR.

Dox is an effective cytotoxic drug targeting newly synthesized DNA in the form of DNA topoisomerase II complex (25, 26). When administered alone, Dox is extremely effective in suppressing MCF-7 growth initially, yet Dox resistance is unavoidable. The inventors have shown that low concentrations of M₄N can be used to suppress MDR-1 gene expression, both in Dox sensitive MCF-7 cells and in Dox resistant NCI/ADR-RES cells. The gene product of MDR-1, the Pgp protein, is commonly known for its ability to expel cytotoxic drugs such as Dox. It is shown in the results detailed above that Pgp can be eliminated following M₄N treatment (FIG. 6). In the absence of Pgp, more Dox should be available at the sites necessary for its cytotoxic activity.

In addition to suppressing MDR gene expression, M₄N exerts independently its control of cell growth at G₂/M phase of the cell cycle. NDGA derivatives together with other anticancer drugs working in concert should offer distinctive advantages in keeping mestastatic tumor growth in check without raising drug resistances and host toxicities consequently.

All patents and publications cited herein are hereby incorporated by reference.

### REFERENCES

1. Hwu, J. R., Tseng, W. N., Gnabre, J., Giza, P. and Huang, R. C. Antiviral activities of methylated nordihydroguaiaretic acids. 1. Synthesis, structural identification and inhibition of Tat-regulated HIV transactivation. J. Med. Chem. 41, 2994-3000, 1998.

2. Chen, H. S., Teng, Li, Li, J. N., Park, R., Mold, D., Gnabre, J., Hwu, J. R., Tseng, W. N. and Huang, R. C. Antiviral activities of methylated nordihydroguaiaretic acids. 2. Targeting herpes simplex virus replication by mutation insensitive transcription inhibiter Tetra-o-methyl-NDGA. J. Med. Chem. 41, 3001-3007,1998.

3. Heller, J. D., Kuo, J., Wu, T. C., Kast, M. and Huang, R. C. Tetra-o-methyl-nordihydroguaiaretic acid induces G₂ arrest in mammalian cells and exhibits tumoricidal activity in vivo. Cancer Res. 61, 5499-5504, 2001.

4. Chang, C. C., Heller, J. D., Kuo, J. and Huang, R. C. Tetra-o-methyl noridhydroguaiaretic acid induces growth arrest and cellular apoptosis by inhibiting Cdc2 and Survivin expression. Proc. Natl. Acad. Sci. 101, 13239-13244, 2004.

5. Park, R., Chang, C. C., Liang, Y. C., Chung, Y., Henry, R. A., Lin, E., Mold, D. and Huang, R. C. Systemic treatment with tetra-o-methyl nordihydroguaiaretic acid suppresses the growth of human xenograft tumors. Clin. Cancer Res. 11(12) 4601-4609, 2005.

6. Huang, R. C., Park, R., Chang, C. C., Liang, Y. C., Mold, D., Lin, E., Heller, J. and Frazer, N. U.S. patent application file May 20, 2004, Ref. No. 1150-0002.40

7. Juliano, R. L. and Ling, V. A surface glycoprotein modulating drug permeability in Chinese hamster ovary cell mutants. Biochem. Biophys. Acta 455, 152-162, 1976.

8. Gottesman, M. M. and Ambudkar, S. V. ABC transporters and human disease. J. Bioenerg. Biomembr. 33, 453-458, 2001.

9. Scotto, K. and Egan, D. A. Transcriptional regulation of MDR genes. Cytotechnology 27, 257-269, 1998.

10. Scotto, K. W. Transcriptional regulation of ABC drug transporter. Oncogene 22, 7496-7511, 2003.

11. Shengkan, J., Gorfajn, B., Faircloth, G. and Scotto, K. W. Ecteinascidin 743, a transcription-targeted chemotherapeutic that inhibits MDR1 activation. Proc. Natl. Acad. Sci. 97, 6775-6779, 2000.

12. Xu, D., Ye, D., Fisher, M. and Juliano, R. L. Selective inhibition of P-glycoprotein expression in multidrug-resistant tumor cells by a designed transcriptional regulator. J. Pharm. & Experimental Therapapeutics 302, 963-971, 2002.

13. Dewick, P. M. and Jackson, D. G. Cytotoxic lignans from podophyllum and the nomenclature of aryltetralin lignans. Cytochemistry 20, 2277, 1981.

14. Gnabre, J. N., Brady, J. N., Clauton, D. J., Ito, Y., Dittmer, J., Bates, R. B. and Huang, R. C. Inhibition of human immunodeficiency virus type 1 transcription and replication by DNA sequence-selective plant lignans. Proc. Natl. Acad. Sci. 92, 11239-11243, 1995.

15. Craigo, J., Callahan, M., Huang, R. C. and Delucia, A. Inhibition of human papillomavirus type 16 gene expression by nordihydroguaiaretic acid plant lignan derivatives. Antiviral Res. 47, 19-28, 2000.

16. Chou T C, Talalay P (1984) Quantitative analysis of dose-effect relationships: the combined effects of multiple drugs or enzyme inhibitors. Adv Enzyme Regul 22:27-55.

17. Chang T T, Chou T C (2000) Rational approach to the clinical protocol design for drug combinations: a review. Acta Paediatr Taiwan 41:294-302

18. Chou T C (1991) In: Chou T C, Rideout D C (eds) Synergism and antagonism in chemotherapy. Academic, San Diego, pp 61-102

19. Chou T C, Motzer R J, Tong Y, Bosl G J (1994) Computerized quantitation of synergism and antagonism of taxol, topotecan, and cisplatin against human teratocarcinoma cell growth: a rational approach to clinical protocol design. J Natl Cancer Inst 86:1517-24

20. Chou T C, Martin N (2005) CompuSyn for Drug Combinations. ComboSyn, Inc. Paramus, N.J.

21. Wolfson, M L and A Thompson (1962), Methods in Carbohydrate Chem., Vol I, p. 334.

22. Chao T C, Chu Z, Tseng L M, Chiou T J, Hsieh R K, Wang W S, Yen C C, Yang M H, Hsiao L T, Liu J H, Chen P M (2005) Paclitaxel in a novel formulation containing less Cremophor EL as first-line therapy for advanced breast cancer: a phase II trial. Invest. New Drugs 23:171-177.

23. Chang C -C: Ph D Thesis, Johns Hopkins University, 2005.

24. Bissery MCm Guenard D, Gueritte-Voegelein F, Lavelle F (1991) Experimental antitumor activity of taxotere (RP 56976, NSC 628503), a taxol analog. Cancer Research 51:4845-4852.

25. Tewey, K. M., Rowe, T. C., Yang, L., Halligan, B. D. and Liu, L. F. Adriamycin-induced DNA damage mediated by mammalian DNA topoisomerase II. Science 226, 466-468, 1984.

26. Nelson, W. G., Liu, L. F. and Coffey, D. S. Newly replicated DNA is associated with DNA topoisomerase II in cultured rat prostatic adenocarcinoma cells. Nature 322, 187-189, 1986.

1-57. (canceled)

58. A method for preventing at least one of synthesis and function of drug transporter protein Pgp in a cell, the method comprising administering an NDGA derivative or a physiologically acceptable salt thereof, the NDGA derivative having the formula 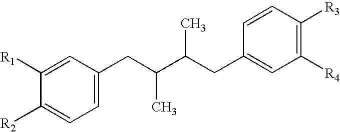
wherein R₁, R₂, R₃ and R₄ are each independently selected from the group consisting of HO—, CH₃O—, CH₃(C═O)O—, an amino acid residue, a substituted amino acid residue and a saccharide residue; the amino acid residue, substituted amino acid residue or saccharide residue being optionally joined to the phenyl ring by a linker selected from the group consisting of at least one of an oxygen atom and 1-10 carbon atoms, with the proviso that
i) at least one of R₁-R₄ comprises an amino acid residue or substituted amino acid residue, or
ii) one of R₁-R₄ comprises a saccharide residue.

59. The method according to claim 58 wherein at least one of R₁-R₄ comprises a residue of an amino acid having at least two —CH₂— groups present between an amino group and a carboxyl group.

60. The method according to claim 59 wherein R₁-R₄ each comprises —CO—(CH₂)₃—N—(CH₃)₂.

61. The method according to claim 58 wherein one of R₁-R₄ comprises a monosaccharide residue or disaccharide residue.

62. The method according to claim 58 wherein at least one of R₁-R₄ comprises an amino acid residue, substituted amino acid residue or saccharide residue, wherein the amino acid residue, substituted amino acid residue or saccharide residue is joined to the phenyl ring by a linker selected from the group consisting of at least one of an oxygen atom and 1-10 carbon atoms.

63. The method according to claim 62 wherein R₁ is —O—CH₂—CH₂-maltose or —O—CH₂—CH₂-galactose, and R₂-R₄ are each —OCH₃.

64. The method according to claim 58 wherein synthesis of Pgp is caused by a chemotherapeutic agent.

65. The method according to claim 64 wherein the chemotherapeutic agent is selected from the group consisting of doxorubicin, vinblastine, paclitaxel, and vincristine.

66. The method according to claim 58 wherein the cell is a tumor cell.

67. The method according to claim 58 wherein the cell is an infectious microorganism.

68. The method according to claim 67 wherein the cell is a virus, bacterium, parasite or fungus.

69. The method according to claim 58 wherein the NDGA derivative prevents chemical induction of multiple drug resistance gene 1 (MDR1) expression in a cell.

70. The method according to claim 58 wherein the NDGA derivative prevents expression of MDR1 in a cell.

71. A method of treating cancer, the method comprising administering an NDGA derivative or physiologically acceptable salt thereof in combination with at least one secondary chemotherapeutic agent to treat cancer, the NDGA derivative having a formula 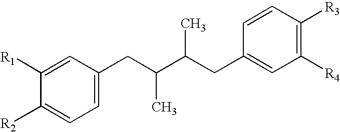
wherein
i) at least one of R₁-R₄ comprises an amino acid residue having at least 2 —CH₂— groups present between an amino and a carboxyl group, or comprises a substituted amino acid residue having at least 2 —CH₂— groups between an amino and a carboxyl group, and the remaining R groups are independently selected from HO—, CH₃O— and CH₃(C═O)O—; or
ii) one of R₁-R₄ comprises a saccharide residue and the remaining R groups are selected from HO—, CH₃O— and CH₃(C═O)O—;
the amino acid residue, substituted amino acid residue or saccharide residue being optionally joined to the phenyl ring by a linker selected from the group consisting of at least one of an oxygen atom and 1-10 carbon atoms.

72. The method according to claim 71 wherein at least one of R₁-R₄ comprises a residue of an amino acid having at least two —CH₂— groups present between the amino group and the carboxyl group.

73. The method according to claim 72 wherein R₁-R₄ each comprises —CO—(CH₂)₃—N—(CH₃)₂.

74. The method according to claim 71 wherein one of R₁-R₄ comprises a monosaccharide residue or disaccharide residue.

75. The method according to claim 74 wherein the monosaccharide or disaccharide residue is joined to the phenyl ring by a linker selected from the group consisting of at least one of an oxygen atom and 1-10 carbon atoms.

76. The method according to claim 75 wherein one of R₁-R₄ is —O—CH₂—CH₂— maltose or —O—CH₂—CH₂-galactose, and the remainder of R₁-R₄ are —OCH₃.

77. The method according to claim 71 wherein the secondary chemotherapeutic agent is selected from the group consisting of doxorubicin, vinblastine, paclitaxel, and vincristine.

78. The method according to claim 71 wherein the cell is a tumor cell.

79. The method according to claim 71 wherein the cell is an infectious microorganism.

80. The method according to claim 79 wherein the cell is a virus, bacterium, parasite or fungus.

81. The method according to claim 71 wherein the molar ratio of the NDGA derivative to the secondary chemotherapeutic agency is about 20:1.

82. The method according to claim 71 wherein the molar ratio of the NDGA derivative to the secondary chemotherapeutic agency is about 2.4:1.

83. A method of preventing or overcoming multiple drug resistance in a cancer cell, the method comprising the administration of an NDGA derivative or physiologically acceptable salt thereof to the cancer cell, the NDGA derivative having a formula 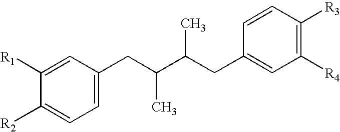
wherein R₁, R₂, R₃ and R₄ are each independently selected from the group consisting of HO—, CH₃O—, CH₃(C═O)O—, an amino acid residue, a substituted amino acid residue and a saccharide residue; the amino acid residue, substituted amino acid residue or saccharide residue being optionally joined to the phenyl ring by a linker selected from the group consisting of at least one of an oxygen atom and 1-10 carbon atoms, with the proviso that
i) at least one of R₁-R₄ comprises an amino acid residue or substituted amino acid residue, or
ii) one of R₁-R₄ comprises a saccharide residue.

84. The method according to claim 83 wherein at least one of R₁-R₄ comprises a residue of an amino acid having at least two —CH₂— groups present between the amino group and the carboxyl group.

85. The method according to claim 83 wherein R₁-R₄ each comprises —CO—(CH₂)₃—N—(CH₃)₂.

86. The method according to claim 83 wherein one of R₁-R₄ comprises a monosaccharide or disaccharide residue.

87. The method according to claim 86 wherein at least one of R₁-R₄ is —O—CH₂—CH₂-maltose or —O—CH₂—CH₂-galactose, and the remainder of R₁-R₄ are —OCH₃.

88. The method according to claim 83, further comprising administering a drug selected from the group consisting of doxorubicin, vinblastine, paclitaxel, and vincristine.

89. The method according to claim 83 wherein the cell is a tumor cell.

90. The method according to claim 89 wherein the tumor cell is a human cancer cell.

91. The method according to claim 90 wherein the cancer is selected from the group consisting of breast cancer, lung cancer, melanoma, ovarian cancer, multiple myeloma, and Non-Hodgkin's Lymphoma.

92. The method according to claim 58 wherein the cell is the cell of a nonhuman animal.

93. The method according to claim 92 wherein the animal is a mammal.

94. The method according to claim 71 wherein the cancer is a cancer of a nonhuman animal.

95. The method according to claim 94 wherein the animal is a mammal.

96. The method according to claim 83 wherein the cell is the cell of a nonhuman animal.

97. The method according to claim 96 wherein the animal is a mammal.

98. A method of overcoming drug resistance in a microorganism, comprising administering to the microorganism or a host containing the microorganism an effective amount of an NDGA derivative or a physiologically acceptable salt thereof, the NDGA derivative having the formula 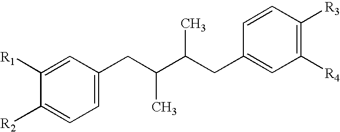
wherein R₁, R₂, R₃ and R₄ are each independently selected from the group consisting of HO—, CH₃ O—, CH₃(C═O)O—, an amino acid residue, a substituted amino acid residue and a saccharide residue; the amino acid residue, substituted amino acid residue or saccharide residue being optionally joined to the phenyl ring by a linker selected from the group consisting of at least one of an oxygen atom and 1-10 carbon atoms; with the proviso that
i) at least one of R₁-R₄ comprises an amino acid residue or substituted amino acid residue, or
ii) one of R₁-R₄ comprises a saccharide residue.

99. The method of claim 98 wherein the microorganism is selected from the group consisting of a virus, a bacterium, a parasite and a fungus.

100. A method of treating a drug-resistant infection in an animal, comprising administering to the animal, along with at least one therapeutic agent to which the infection is resistant, an effective amount of a compound, or a physiologically acceptable salt thereof, of formula 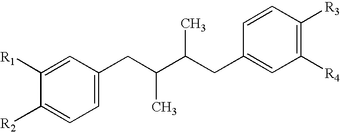
wherein R₁, R₂, R₃ and R₄ are each independently selected from the group consisting of HO—, CH₃O—, CH₃(C═O)O—, an amino acid residue, a substituted amino acid residue and a saccharide residue; the amino acid residue, substituted amino acid residue or saccharide residue being optionally joined to the phenyl ring by a linker selected from the group consisting of at least one of an oxygen atom and 1-10 carbon atoms, with the proviso that
i) at least one of R₁-R₄ comprises an amino acid residue or substituted amino acid residue, or
ii) one of R₁-R₄ comprises a saccharide residue.

101. The method of claim 100 wherein the infection is a viral, bacterial, parasitic or fungal infection.

102. A composition comprising a compound of formula 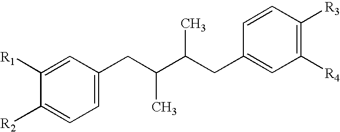
or a physiologically acceptable salt thereof, wherein
i) at least one of R₁-R₄ comprises a β-amino acid residue linked to the phenyl ring through an oxygen atom; or
ii) one of R₁-R₄ comprises a saccharide residue, optionally linked to the phenyl ring through an oxygen atom and 1-10 —CH₂— groups; and
the remainder of R₁-R₄ are —OCH₃.

103. The composition of claim 102 wherein one of R₁-R₄ comprises a monosaccharide residue or disaccharide residue.

104. The composition of claim 103 wherein the saccharide residue is selected from the group consisting of a residue of glucose, galactose, mannose, fucose, glucosamine, galactosamine and derivatives thereof on which the —OH groups or amino groups are modified by attachment of or replacement with substituents selected from O-methyl, O-acetyl, amino, carboxyl, lower alkyl, lower acyl, phospho and sulfo groups.

105. The composition of claim 102 wherein one of R₁-R₄ comprises an oligosaccharide residue consisting of at least two sugars of the same or different kinds.

106. The composition of claim 103 wherein the compound is maltose-M₃N or galactose-M₃N.

107. The composition of claim 102 wherein at least one of R₁-R₄ comprises a amino acid residue.

108. The composition of claim 102 comprising 5-((2S,3R)-4-{3,4-bis[4-(dimethylamino)butanoyloxy]phenyl}-2,3-dimethylbutyl)-2-[4-(dimethylamino)butanoyloxy]phenyl 4-(dimethylamino)butanoate or 5-((2S,3R)-4-{3,4-bis[4-(dimethylamino)propanoyloxy]phenyl}-2,3-dimethylbutyl)-2-[4-(dimethylamino)propanoyloxy]phenyl 4-(dimethylamino)propanoate.

109. The composition of claim 102 that additionally comprises a secondary chemotherapeutic agent.

110. The composition of claim 109 wherein the secondary chemotherapeutic agent is selected from the group consisting of doxorubicin, vinblastine, paclitaxel, and vincristine.

111. The composition of claim 109 wherein the NDGA derivative and the secondary chemotherapeutic agent have a molar ratio of about 2:1 to about 100:1.

112. A composition comprising an NDGA derivative, or a physiologically acceptable salt thereof, and a secondary chemotherapeutic agent, the NDGA derivative having the formula 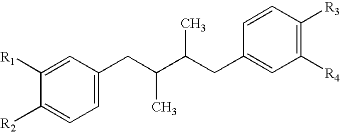
wherein
i) at least one of R₁-R₄ comprises an amino acid residue having at least 2 —CH₂— groups present between an amino and a carboxyl group, or comprises a substituted amino acid residue having at least 2 —CH₂— groups between an amino and a carboxyl group, and the remaining R groups are independently selected from HO—, CH₃O— and CH₃(C═O)O—; or
ii) one of R₁-R₄ comprises a saccharide residue and the remaining R groups are selected from HO—, CH₃O— and CH₃(C═O)O—;
the amino acid residue, substituted amino acid residue or saccharide residue being optionally joined to the phenyl ring by a linker selected from the group consisting of at least one of an oxygen atom and 1-10 carbon atoms.

113. The composition of claim 112 wherein the NDGA derivative and the secondary chemotherapeutic agent have a molar ratio of about 2:1 to about 100:1.

114. The composition of claim 112 comprising M₄N or maltose-M₃N and paclitaxel in a molar ratio of about 20:1.

115. The composition of claim 112 comprising M₄N or maltose-M₃N and doxorubicin in a molar ratio of about 2.4:1.

116. A method for determining an optimum dosage combination of an NDGA derivative, or a physiologically acceptable salt thereof, and a secondary chemotherapeutic agent for treating a cancer, the NDGA derivative having the formula 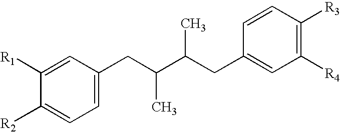
wherein R₁, R₂, R₃ and R₄ are each independently selected from the group consisting of HO—; CH₃O—; CH₃(C═O)O—; an amino acid residue; a substituted amino acid residue; a saccharide residue; the amino acid residue, substituted amino acid residue or saccharide residue being optionally joined to the phenyl ring by a linker selected from the group consisting of at least one of an oxygen atom and 1-10 carbon atoms; the method comprising
(a) administering a series of compositions of varying dosages of the NDGA derivative and the secondary chemotherapeutic agent to a culture of cancer cells;
(b) measuring the growth rate of the cells;
(c) using an isobologram method or combination index method to determine the optimal combination dosage to achieve comparable efficacy with suboptimal concentrations for both the NDGA derivative and the secondary chemotherapeutic agent.

117. A composition comprising the optimal dosage combination of claim 116.

118. A method of treating cancer comprising administration of an effective amount of the composition of claim 117 to an individual in need of treatment.


SEQUENCE LISTING (4 sequences)

SEQ ID NO: 1 - DNA, 20 bp, Artificial Sequence
  Description of Artificial Sequence Synthetic primer
acatgaccaggtatgcctat

SEQ ID NO: 2 - DNA, 20 bp, Artificial Sequence
  Description of Artificial Sequence Synthetic primer
gaagatagtatctttgccca

SEQ ID NO: 3 - DNA, 20 bp, Artificial Sequence
  Description of Artificial Sequence Synthetic primer
ccatcaccatcttccaggag

SEQ ID NO: 4 - DNA, 20 bp, Artificial Sequence
  Description of Artificial Sequence Synthetic primer
cctgcttcaccaccttcttg